SEQUENCE LISTING (40 sequences)

SEQ ID NO: 1 - protein, 50 aa, synthetic construct
  REGION 1..50: note = FTVPP HSPYV HTTTR FRFWD RPDMD ARWFR YYRHH RYFRW YRFKR FFRRY
FTVPPHSPYVHTTTRFRFWDRPDMDARWFRYYRHHRYFRWYRFKRFFRRY

SEQ ID NO: 2 - protein, 50 aa, synthetic construct
  REGION 1..50: note = PSWEN PHSPY KFRRF FRYFR WRWYY PSDED AEFHH RHHHR RYYRH RYVRD
PSWENPHSPYKFRRFFRYFRWRWYYPSDEDAEFHHRHHHRRYYRHRYVRD

SEQ ID NO: 3 - protein, 50 aa, synthetic construct
  REGION 1..50: note = QYLTG RDEDE ELSNE DKERH HRYYR YFRWW RWRDY WLRFH HHDNT VPTQQ
QYLTGRDEDEELSNEDKERHHRYYRYFRWWRWRDYWLRFHHHDNTVPTQQ

SEQ ID NO: 4 - protein, 42 aa, synthetic construct
  REGION 1..42: note = WNKKF YFRRF FWDDS FHHHT RTWTV FHRHR TYRLT TDRGR FH
WNKKFYFRRFFWDDSFHHHTRTWTVFHRHRTYRLTTDRGRFH

SEQ ID NO: 5 - protein, 48 aa, synthetic construct
  REGION 1..48: note = QSEPSWMFAAQVSELFFRFRATDYRHRHHPDLVENKFRHSPYDRPDRY
QSEPSWMFAAQVSELFFRFRATDYRHRHHPDLVENKFRHSPYDRPDRY

SEQ ID NO: 6 - protein, 49 aa, synthetic construct
  REGION 1..49: note = AQDETYYRYDRYDDSDTNTRDEDRPLSWPHSDPYYWLRHYHRLMETLQV
AQDETYYRYDRYDDSDTNTRDEDRPLSWPHSDPYYWLRHYHRLMETLQV

SEQ ID NO: 7 - protein, 48 aa, synthetic construct
  REGION 1..48: note = ETYEMQDTTRQKLEELRRFFDPYRFRSEVSYHRDMFAQDDTRSFEDAR
ETYEMQDTTRQKLEELRRFFDPYRFRSEVSYHRDMFAQDDTRSFEDAR

SEQ ID NO: 8 - protein, 48 aa, synthetic construct
  REGION 1..48: note = DARDRYEWKELWEESYDTAQDHWWRWYEMQRWWVHTEKKEMETRDTVR
DARDRYEWKELWEESYDTAQDHWWRWYEMQRWWVHTEKKEMETRDTVR

SEQ ID NO: 9 - protein, 48 aa, synthetic construct
  REGION 1..48: note = PAYRFKYTDAITDDYWNKPSWLEELYWLRDTTRKQVRGRHPDWSPEDR
PAYRFKYTDAITDDYWNKPSWLEELYWLRDTTRKQVRGRHPDWSPEDR

SEQ ID NO: 10 - protein, 50 aa, synthetic construct
  REGION 1..49: note = DDYEDRHPDWFRYLEEWSPDPYQSEERKEEMQYRYDTTMTALSFDNTRWR
DDYEDRHPDWFRYLEEWSPDPYQSEERKEEMQYRYDTTMTALSFDNTRWR

SEQ ID NO: 11 - protein, 49 aa, synthetic construct
  REGION 1..48: note = DTVWNKWFHRYHRWFRWWRAITYPSDTRHWWQSEEYLFSTDSDTKQDNT
DTVWNKWFHRYHRWFRWWRAITYPSDTRHWWQSEEYLFSTDSDTKQDNT

SEQ ID NO: 12 - protein, 48 aa, synthetic construct
  REGION 1..48: note = TLTEDKHWWATDWSPERLFSTYTDHYHRWWTWRPAYTFTYPSTQQDMT
TLTEDKHWWATDWSPERLFSTYTDHYHRWWTWRPAYTFTYPSTQQDMT

SEQ ID NO: 13 - protein, 48 aa, synthetic construct
  REGION 1..48: note = RGRRDEVHTHYHTQQYTDRFWFHRETRFYDPAYFSTAITFLEDTRMET
RGRRDEVHTHYHTQQYTDRFWFHRETRFYDPAYFSTAITFLEDTRMET

SEQ ID NO: 14 - protein, 48 aa, synthetic construct
  REGION 1..48: note = DDYDDSRRYWFHMENEFYDTVETRQFLRFKQFVTTRKYENLEDTKKMI
DDYDDSRRYWFHMENEFYDTVETRQFLRFKQFVTTRKYENLEDTKKMI

SEQ ID NO: 15 - protein, 50 aa, synthetic construct
  REGION 1..50: note = AHAWYYFYHWYHHLYRHFYDSRHWHFHYFDSTHTSHRIHSDEDDERHPRF
AHAWYYFYHWYHHLYRHFYDSRHWHFHYFDSTHTSHRIHSDEDDERHPRF

SEQ ID NO: 16 - protein, 48 aa, synthetic construct
  REGION 1..48: note = RFVRPRHLFPRFQHVRDSDEDDEAQDATTSRHIRYWQVRNRYMQRPAV
RFVRPRHLFPRFQHVRDSDEDDEAQDATTSRHIRYWQVRNRYMQRPAV

SEQ ID NO: 17 - protein, 48 aa, synthetic construct
  REGION 1..48: note = DTSFRYWHYFATITTEYHWHFYHHHRIHFRPFRNYERDDARVFMRWYH
DTSFRYWHYFATITTEYHWHFYHHHRIHFRPFRNYERDDARVFMRWYH

SEQ ID NO: 18 - protein, 50 aa, synthetic construct
  REGION 1..50: note = RPHRPWLHVRIRYWRMHFVRMQRFYHWYHHSSHDARVPAVFHYFWHHQVR
RPHRPWLHVRIRYWRMHFVRMQRFYHWYHHSSHDARVPAVFHYFWHHQVR

SEQ ID NO: 19 - protein, 50 aa, synthetic construct
  REGION 1..50: note = QPTPRHPRYRTTSFMRHTSDDDEDDTPTPHRVHRPHTYSPAVDSDEFRPF
QPTPRHPRYRTTSFMRHTSDDDEDDTPTPHRVHRPHTYSPAVDSDEFRPF

SEQ ID NO: 20 - protein, 49 aa, synthetic construct
  REGION 1..49: note = HMRRNFISQAYAPRYDDTPTSSTHTSWHHRLYRDYEDHVRASWDSRFRY
HMRRNFISQAYAPRYDDTPTSSTHTSWHHRLYRDYEDHVRASWDSRFRY

SEQ ID NO: 21 - protein, 49 aa, synthetic construct
  REGION 1..49: note = AYADDDTSDAARTTDHIRYSYSTHTHNSARVPFRPHRPFDSRHRIHTQQ
AYADDDTSDAARTTDHIRYSYSTHTHNSARVPFRPHRPFDSRHRIHTQQ

SEQ ID NO: 22 - protein, 48 aa, synthetic construct
  REGION 1..48: note = SYSFRNYQPTFVRAYDTTSRWFDYERMHWHFYAMRMTATYSEMQRLHV
SYSFRNYQPTFVRAYDTTSRWFDYERMHWHFYAMRMTATYSEMQRLHV

SEQ ID NO: 23 - protein, 50 aa, synthetic construct
  REGION 1..50: note = EMQRHLRNFERDATIISQAMRDSTHTTEPRHPRFQHRVFRNYDNTTDPRY
EMQRHLRNFERDATIISQAMRDSTHTTEPRHPRFQHRVFRNYDNTTDPRY

SEQ ID NO: 24 - protein, 48 aa, synthetic construct
  REGION 1..48: note = AHAATTDAQDDHVDTTEFHYRNFDDDATDWYYSSTEMQHRVPFRYRWF
AHAATTDAQDDHVDTTEFHYRNFDDDATDWYYSSTEMQHRVPFRYRWF

SEQ ID NO: 25 - protein, 49 aa, synthetic construct
  REGION 1..49: note = RHLQVRFPRFHYQPTDSDATTNRYRMHHNSSYSFMRTQQDSTHWYYDTT
RHLQVRFPRFHYQPTDSDATTNRYRMHHNSSYSFMRTQQDSTHWYYDTT

SEQ ID NO: 26 - protein, 50 aa, synthetic construct
  REGION 1..50: note = RFQRPWDNTTSSTMTARTTRLYRDQDDAAASWFPRNRYTPTAMRRFVRHI
RFQRPWDNTTSSTMTARTTRLYRDQDDAAASWFPRNRYTPTAMRRFVRHI

SEQ ID NO: 27 - protein, 48 aa, synthetic construct
  REGION 1..48: note = ERDHIRHMRTQQASWATIHNSAQDDHVAYDDTTLHVRHIDTSSSHDQD
ERDHIRHMRTQQASWATIHNSAQDDHVAYDDTTLHVRHIDTSSSHDQD

SEQ ID NO: 28 - protein, 48 aa, synthetic construct
  REGION 1..48: note = TYSMTASSHDAARWFNTTATDRPWDNTWHHDYEHWHAYARLYDTPHMR
TYSMTASSHDAARWFNTTATDRPWDNTWHHDYEHWHAYARLYDTPHMR

SEQ ID NO: 29 - protein, 48 aa, synthetic construct
  REGION 1..48: note = ISQDARDQDRFVATDAYDAHAMNMKPIIMAQWRPQQPDPMELRQNYID
ISQDARDQDRFVATDAYDAHAMNMKPIIMAQWRPQQPDPMELRQNYID

SEQ ID NO: 30 - protein, 50 aa, synthetic construct
  REGION 1..50: note = FREWNKFRRF SLFFSTFWRL EFPFSHPDFR FLPQTIPRFR FWLRDRIWS
FREWNKFRRFSLFFSTFWRLEFPFSHPDFRFLPQTIPRFRFWLRDRIWSP

SEQ ID NO: 31 - protein, 48 aa, synthetic construct
  REGION 1..48: note = HFHFRNWEVH HHDTRHRHWF RDPYPSWQLP FISRDEDTVH SPSVHETY
HFHFRNWEVHHHDTRHRHWFRDPYPSWQLPFISRDEDTVHSPSVHETY

SEQ ID NO: 32 - protein, 48 aa, synthetic construct
  REGION 1..48: note = GGAAGGHPDG GAAGGFHSGG AAGGDPYGGA AGGDTRGGAA GGHHHHGG
GGAAGGHPDGGAAGGFHSGGAAGGDPYGGAAGGDTRGGAAGGHHHHGG

SEQ ID NO: 33 - protein, 48 aa, synthetic construct
  REGION 1..48: note = GGAAGGHSPG GAAGGDFPGG AAGGYFSGGA AGGETHGGAA GGHHHHGG
GGAAGGHSPGGAAGGDFPGGAAGGYFSGGAAGGETHGGAAGGHHHHGG

SEQ ID NO: 34 - protein, 32 aa, synthetic construct
  REGION 1..32: note = GGAAGGHPDG GFHSGGDPYG GDTRGGHHHH GG
GGAAGGHPDGGFHSGGDPYGGDTRGGHHHHGG

SEQ ID NO: 35 - protein, 32 aa, synthetic construct
  REGION 1..32: note = GGAAGGHSPG GDFPGGVFSG GETHGGHHHH GG
GGAAGGHSPGGDFPGGVFSGGETHGGHHHHGG

SEQ ID NO: 36 - protein, 32 aa, synthetic construct
  REGION 1..32: note = GGAAGGDPYG GDTRGGHPDG GFHSGGHHHH GG
GGAAGGDPYGGDTRGGHPDGGFHSGGHHHHGG

SEQ ID NO: 37 - protein, 32 aa, synthetic construct
  REGION 1..32: note = GGAAGGYFSG GETHGGHSPG GDFPGGHHHH GG
GGAAGGYFSGGETHGGHSPGGDFPGGHHHHGG

SEQ ID NO: 38 - protein, 32 aa, synthetic construct
  REGION 1..32: note = GGAAGGHSPGGDFPGGDPYGGDTRGGHHHHGG
GGAAGGHSPGGDFPGGDPYGGDTRGGHHHHGG

SEQ ID NO: 39 - protein, 32 aa, synthetic construct
  REGION 1..32: note = GGAAGGHPDGGFHSGGYFSGGETHGGHHHHGG
GGAAGGHPDGGFHSGGYFSGGETHGGHHHHGG

SEQ ID NO: 40 - protein, 32 aa, synthetic construct
  REGION 1..32: note = KGKAGGHSPGGDFPGGYFSGGFHSGGHHHHGG
KGKAGGHSPGGDFPGGYFSGGFHSGGHHHHGG

(19) United States
(12) Patent Application Publication
(10) Pub. No.: US 2024/0183858 A1
(43) Pub. Date: Jun. 6, 2024

(54) STANDARD POLYPEPTIDES

(71) Applicant: Nautilus Subsidiary, Inc., Seattle, WA (US)

(72) Inventors: Hunter Bryan BOYCE, Union City, CA (US); Kara JUNEAU, San Carlos, CA (US); Torri Elise RINKER, San Francisco, CA (US); Parag MALLICK, San Mateo, CA (US)

(21) Appl. No.: 18/510,429

(22) Filed: Nov. 15, 2023

Related U.S. Application Data
(60) Provisional application No. 63/383,868, filed on Nov. 15, 2022.
(60) Provisional application No. 63/385,721, filed on Dec. 1, 2022.

(51) Int. Cl. G01N 33/68; C07K 1/22; C07K 14/00; G01N 33/53

(52) U.S. Cl. CPC G01N 33/6842; C07K 1/22; C07K 14/001; G01N 33/5308; G01N 2570/00

(57) ABSTRACT

A set of polypeptides including a plurality of different polypeptides, each of the different polypeptides including a non-naturally occurring amino acid sequence, wherein a set of different epitopes occurs in the set of polypeptides, each of the different epitopes occurring in the non-naturally occurring amino acid sequence of a subset of the different polypeptides, and the non-naturally occurring amino acid sequence of each of the different polypeptides including a plurality of different epitopes of the set of epitopes.

### CROSS-REFERENCE TO RELATED APPLICATIONS

This application claims priority to U.S. Provisional Application No. 63/385,721, filed on Dec. 1, 2022, and U.S. Provisional Application No. 63/383,868, filed on Nov. 15, 2022, each of which is incorporated herein by reference in its entirety.

### SEQUENCE LISTING

The instant application contains a Sequence Listing which has been submitted electronically in XML format and is hereby incorporated by reference in its entirety. The XML copy, created on Nov. 15, 2023, is named NBIOT.020SeqListing.xml and is 53,309 bytes in size.

### BACKGROUND

The proteome is a dynamic and valuable source of biological insight and clinical diagnosis. Despite the wealth of insights gained from now routine genomics and transcriptomics studies in biomedical research, a large gap remains between genome/transcriptome and phenotype. Proteomics is crucial to bridging this gap since the polypeptides that constitute the proteome are the main structural and functional components that drive an individual's phenotype. Technologies for identifying and characterizing polypeptides at scales that match the complexity of a typical proteome lag behind DNA sequencing technologies. This is due, at least in part, to the increased variability of biochemical properties for polypeptides compared to DNA, which make them more difficult to process in multiplexed assays, and the significantly larger dynamic range in the quantities of different polypeptides present in a cell at any given time compared to DNA or RNA in the same cell which challenges the detection range for detectors and assays that have been designed for nucleic acids. Moreover, a substantial number of the polypeptides predicted to comprise the human proteome have not been confidently observed to date.

Recently, binding assays have been designed for identifying large sets of polypeptides, for example, at proteome scale. See for example, U.S. Pat. Nos. 10,473,654 or 11,282,585; US Pat App. Pub. No. 2023/0114905 A1; or Egertson et al., BioRxiv (2021), DOI: 10.1101/2021.10.11.463967, each of which is incorporated herein by reference. The assays utilize affinity reagents having unique properties. Understanding and characterizing these properties is fundamental to obtaining accurate results from the binding assays in which the affinity reagents are used.

### SUMMARY

Described herein are compositions that include one or more selected polypeptides having one or more desired epitopes within their sequence and/or structure. These compositions may serve a variety of purposes. For example, the polypeptides can function as internal controls or standards for assays that detect binding of the epitope(s) to one or more affinity reagents. In other examples, the polypeptides can be used as bait, controls or standards for preparing, modifying or purifying affinity reagents that recognize the epitopes.

The present disclosure provides a set of different polypeptides (e.g. standard polypeptides), wherein a set of different epitopes occurs in the set of different polypeptides. Optionally, the set of different polypeptides is a non-naturally occurring set of polypeptides. In some cases, the set is non-naturally occurring by virtue of including at least one polypeptide having a non-naturally occurring amino acid sequence. Alternatively or additionally, the set is non-naturally occurring by virtue of including two or more polypeptides that do not co-occur in nature. A set of polypeptides that does not co-occur in nature can include, for example, polypeptides that do not co-occur in the same subcellular compartment, cell, tissue, biological fluid, or organism.

In a first configuration, individual polypeptides of a set of different polypeptides can each include a non-naturally occurring amino acid sequence, wherein a set of different epitopes occurs in the set of different polypeptides, each of the different epitopes occurring in the non-naturally occurring amino acid sequence of a subset of the different polypeptides, and the non-naturally occurring amino acid sequence of each of the different polypeptides including a plurality of different epitopes of the set of epitopes. In a second configuration, a subset of the individual polypeptides of a set of different polypeptides can each include a non-naturally occurring amino acid sequence, wherein a set of different epitopes occurs in the set of different polypeptides, each of the different epitopes occurring in the different polypeptides, and each of the different polypeptides including a plurality of different epitopes of the set of epitopes.

The present disclosure provides a set of at least 3 different polypeptides having amino acid sequences of at least 10 amino acids, wherein a set of at least 10 different epitopes occurs in the set of different polypeptides, each of the different epitopes including at least 3 amino acids in the amino acid sequences of a subset of at least 2 of the different polypeptides, and wherein the amino acid sequences of the different polypeptides each includes a subset of at least 3 epitopes different epitopes of the set of epitopes.

The present disclosure also provides a set of polypeptides (e.g. standard polypeptides) including a plurality of different polypeptides, each of the different polypeptides optionally including a non-naturally occurring amino acid sequence, wherein a set of different epitopes occurs in the set of polypeptides, each of the different epitopes occurring in the (optionally non-naturally occurring) amino acid sequence of a subset of the different polypeptides, and the (optionally non-naturally occurring) amino acid sequence of each of the different polypeptides including a plurality of different epitopes of the set of epitopes.

In some configurations, a set of polypeptides (e.g. standard polypeptides) can include at least 2 different polypeptides, each of the different polypeptides including a sequence of at least 6 amino acids, wherein the sequence of at least 6 amino acids in each polypeptide of the different polypeptides is optionally non-naturally occurring, wherein a set of epitopes occurs in the different polypeptides, the set of epitopes including at least 3 different epitopes, each of the epitopes including 3 contiguous amino acids, wherein each of the different epitopes in the set occurs in the sequence of at least 6 amino acids for at least 2 of the different polypeptides, and wherein the sequence of at least 6 amino acids for each of the different polypeptides includes at least 2 different epitopes of the set.

The present disclosure provides a standard polypeptide having the amino acid sequence of any one of SEQ ID NOs: 1 to 40. Also provided is a set of standard polypeptides including at least two amino acid sequences selected from SEQ ID NOs: 1 to 40.

The present disclosure provides a method of preparing a polypeptide sample. The method can include steps of (a) obtaining a polypeptide extract from an organism; and (b) contacting the polypeptide extract with a set of standard polypeptides, thereby forming a polypeptide sample including polypeptides from the extract and the at least one standard polypeptide.

The present disclosure provides a method of detecting polypeptides. The method can include steps of (a) obtaining a sample including a set of standard polypeptides and a plurality of test polypeptides from an organism; and (b) detecting at least one polypeptide from the organism in the sample and detecting the at least one standard polypeptide in the sample.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF THE DRAWINGS

FIG. 1A shows a graph structure for adding an edge to a graph for an epitope list that includes AGM, GMA and GMK with an epitope length of 3 and overlap of 2.

FIG. 1B shows addition of the NAV epitope as a singleton to the graph of FIG. 1A.

FIG. 1C shows a graph that would result if a FASTA file is parsed and transitions between epitopes are counted resulting in the sequence AGMA being observed 8 times and AGMK never being observed.

FIG. 2A shows a graph for a basic traversal.

FIG. 2B shows the basic traversal graph when the GMA node is selected.

FIG. 2C shows the graph when the edge weights for all outgoing edges are used to generate a probability of traversing any of the edges and then an edge is selected based on the probabilities.

FIG. 2D shows the graph when stochastically the edge to node ALL is chosen.

FIG. 2E shows a jump when the graph-based algorithm has no outgoing edges and a random node is selected to continue.

FIG. 3A shows the sequence for the Epi 4 standard polypeptide (SEQ ID NO:4) and indicates the locations of 5 epitopes targeted by the Lobes using underlines.

FIG. 3B shows a box plot of binding rates observed for 30 cycles of a polypeptide binding assay.

FIG. 4A plots the log10 likelihood ratio for polypeptides on an array being identified (“ID”) as Epi 4 (correct) vs. Epi 5 (incorrect) standard polypeptide for each of 30 decoded cycles.

FIG. 4B shows the a priori binding probability for Lobes binding to either the Epi 4 or Epi 5 standard polypeptides and also shows observed binding in binary values with “1” indicating binding observed above a threshold value and “0” indicating lack of binding above the threshold value.

### DETAILED DESCRIPTION

The present disclosure provides compositions that include one or more polypeptides that can be used for any of a variety of different purposes, including as standard polypeptides to evaluate and characterize affinity reagents and/or to facilitate processes that employ such affinity reagents. Also provided are methods, systems and apparatus that employ and/or incorporate such polypeptide compositions. A standard polypeptide included within a composition set forth herein may include one or more epitopes that serve as binding targets for one or more affinity reagents of interest. A set of standard polypeptides can be configured to include multiple different polypeptides and each of the different polypeptides can contain multiple different epitopes. Moreover, one or more epitopes can be redundantly present across multiple different polypeptides in a set of standard polypeptides. For example, a particular epitope can be present in some or all different polypeptide members of a set of standard polypeptides. A set of standard polypeptides can advantageously provide a rich and compact collection of epitopes for characterizing binding behavior for a plurality of different affinity reagents. This can be especially advantageous when using promiscuous affinity reagents, which recognize relatively small epitopes that are each likely to be present in a variety of different polypeptides, and when performing an assay in which different test polypeptides are contacted with a series of different affinity reagents that produces a pattern of binding which distinguishes the different test polypeptides from each other. As such, the standard polypeptides provide a useful benchmark when assayed and decoded in parallel with test polypeptides.

A polypeptide or set of polypeptides set forth herein can be used in any of a variety of contexts. A particularly useful context is a polypeptide binding assay, wherein one or more polypeptides can be used as standard polypeptide(s) to evaluate activity of one or more affinity reagents used in the assay. For example, a standard polypeptide can serve as a positive or negative control for one or more affinity reagents used in an assay. A set of standard polypeptides can provide a plurality of positive and/or negative controls for binding strength or binding specificity of a set of affinity reagents. Similarly, a standard polypeptide can serve as a quantitation standard for quantifying one or more test polypeptides detected in an assay. For example, standard polypeptides can be provided in known amounts to an assay for test polypeptides, the standard polypeptides and test polypeptides can be quantified, and the quantity of test proteins detected can be determined relative to the known amount of standard polypeptides provided to the assay. In some cases, one or more standard polypeptides can be provided as a series of different amounts and a standard curve can be generated from observed binding of affinity reagents to the series. The standard curve can be used to quantify test proteins detected using the affinity reagents.

Another context in which polypeptides of the present disclosure can be useful is preparation of affinity reagents. For example, a polypeptide (e.g. standard polypeptide) can serve as a target or bait for capturing an affinity reagent of interest in a selection or screening process. Alternatively, one or more polypeptides (e.g. standard polypeptides) can be used in a negative selection step to remove or avoid affinity reagents having unwanted affinity for particular polypeptide structures. In another example, a fluid that contains an affinity reagent can be contacted with an immobilized polypeptide (e.g. standard polypeptide) and affinity reagent that binds the immobilized polypeptide can be separated from the fluid. Separation can occur, for example, via affinity chromatography or solid-phase extraction. Similarly, an affinity reagent can be bound to a labeled polypeptide (e.g. labeled standard polypeptide) to form a labeled complex and the label can be detected to monitor partitioning of the complex in one or more steps of a separation process.

In yet another context, one or more polypeptides (e.g. standard polypeptides) can be used to characterize or assess quality of one or more affinity reagents. For example, binding of an affinity reagent to one or more polypeptides can be evaluated to determine epitope-binding specificity of the affinity reagent, probability of an affinity reagent binding particular epitope(s), strength of affinity reagent binding to particular epitope(s) (e.g. equilibrium dissociation constant or equilibrium association constant), kinetics of affinity reagent binding to particular epitope(s) (e.g. association rate, dissociation rate, kₒₙ or k_{off}). In some cases, specificity of an affinity reagent can be determined based on observed binding (or non-binding) to a set of polypeptides having a plurality of different epitopes.

The present disclosure also provides methods for generating amino acid sequences for a set of polypeptides (e.g. standard polypeptides). Also provided are methods for using polypeptides (e.g. standard polypeptides) in various assay formats. Further provided are sets of polypeptides (e.g. standard polypeptides), for example, immobilized on solid supports, arrays and/or particles. Polypeptides (e.g. standard polypeptides) of the present disclosure can be provided in flow cells, detection instruments, kits, cartridges or arrays, for example, as set forth in further detail herein.

Terms used herein will be understood to take on their ordinary meaning in the relevant art unless specified otherwise. Several terms used herein and their meanings are set forth below.

As used herein, the term “address” refers to a location in an array where a particular analyte (e.g. polypeptide) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces.

As used herein, the term “affinity reagent” refers to a molecule or other substance that is capable of specifically or reproducibly binding to an analyte (e.g. polypeptide). An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to polypeptides include, but are not limited to, antibodies or functional fragments thereof (e.g., Fab' fragments, F(ab')₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

As used herein, the term “array” refers to a population of analytes (e.g. polypeptides) that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include separate solid supports or separate addresses that each bear a different analyte, wherein the different analytes can be identified according to the locations of the solid supports or addresses.

As used herein, the term “attached” refers to the state of two things being joined, fastened, adhered, connected or bound to each other. Attachment can be covalent or non-covalent. For example, a particle can be attached to a polypeptide by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “binding affinity” or “affinity” refers to the strength or extent of binding between an affinity reagent and a binding partner. A binding affinity of an affinity reagent for a binding partner may be qualified as being a “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

The term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a polypeptide or other analyte. Epitopes may include amino acid sequences that are contiguous in the primary structure of a polypeptide. Epitopes may include amino acids that are structurally adjacent in the secondary, tertiary or quaternary structure of a polypeptide despite being non-contiguous in the primary sequence of the polypeptide. An epitope can be, or can include, a moiety of polypeptide that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope need not necessarily participate in, nor be capable of, eliciting an immune response. In some contexts, an epitope that is intended, designed, known, suspected or observed to bind one or more affinity reagents of interest can be referred to as an “epitope for” the one or more affinity reagents of interest or as a “target epitope” of the one or more affinity reagents of interest.

As used herein, the term “exogenous,” when used in reference to a moiety of a molecule, means the moiety is not present in a natural analog of the molecule. For example, an exogenous label of an amino acid is a label that is not present on a naturally occurring amino acid. Similarly, an exogenous label that is present on an antibody is not found on the antibody in its native milieu.

As used herein, the term “fluid-phase,” when used in reference to a molecule, means the molecule is in a state wherein it is mobile in a fluid, for example, being capable of diffusing through the fluid.

As used herein, the term “moiety” refers to a component or part of a molecule. The term does not necessarily denote the relative size of the component or part compared to the rest of the molecule, unless indicated otherwise.

As used herein, the term “immobilized,” when used in reference to a molecule that is in contact with a fluid-phase, refers to the molecule being prevented from diffusing in the fluid-phase. For example, immobilization can occur due to the molecule being confined at, or attached to, a solid support. Immobilization can be temporary (e.g. for the duration of one or more steps of a method set forth herein) or permanent. Immobilization can be reversible or irreversible under conditions utilized for a method, apparatus or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “origami,” when used in reference to a nucleic acid, refers to a construct of the nucleic acid having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structure of the origami. A nucleic acid origami may include sections of single-stranded or double-stranded nucleic acid, or combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “post-translational modification” refers to a change to the chemical composition of a polypeptide compared to the chemical composition encoded by the gene for the polypeptide. Exemplary changes include those that alter the presence, absence or relative arrangement of different regions of amino acid sequence (e.g., splicing variants, or protein processing variants of a single gene), or due to presence or absence of different moieties on particular amino acids (e.g., post-translationally modified variants of a single gene). A post-translational modification can be derived from an in vivo process or in vitro process. A post-translational modification can be derived from a natural process or a synthetic process. Exemplary post-translational modifications include those classified by the PSI-MOD ontology. See Smith, L. M. et al. Nat. Methods, 2013, 10, 186-187.

As used herein, the term “polypeptide” refers to a molecule comprising two or more amino acids joined by a peptide bond. A polypeptide may also be referred to as a protein, oligopeptide or peptide. A polypeptide can be a naturally-occurring molecule, or synthetic molecule. A polypeptide may include one or more non-natural amino acids, modified amino acids, or non-amino acid linkers. A polypeptide may contain D-amino acid enantiomers, L-amino acid enantiomers or both. Amino acids of a polypeptide may be modified naturally or synthetically, such as by post-translational modifications. In some circumstances, different polypeptides may be distinguished from each other based on different genes from which they are expressed in an organism, different primary sequence length or different primary sequence composition. Polypeptides expressed from the same gene may nonetheless be different proteoforms, for example, being distinguished based on non-identical length, non-identical amino acid sequence or non-identical post-translational modifications. Different polypeptides can be distinguished based on one or both of gene of origin and proteoform state.

As used herein, the term “single,” when used in reference to an object such as a polypeptide, means that the object is individually manipulated or distinguished from other objects. Reference herein to a “single analyte” in the context of a composition, apparatus or method herein does not necessarily exclude application of the composition, apparatus or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as polypeptides, reactive moieties, or detectable labels) or are configured to be attached to other molecules (e.g., by functional groups). Exemplary SNAPs include nucleic acid origami and nucleic acid nanoballs.

As used herein, the term “unique identifier” refers to a moiety, object or substance that is associated with an analyte and that is distinct from other identifiers, throughout one or more steps of a process. The moiety, object or substance can be, for example, a solid support such as a particle or bead; a location on a solid support; an address in an array; a tag; a label such as a luminophore; a molecular barcode such as a nucleic acid having a unique nucleotide sequence or a polypeptide having a unique amino acid sequence; or an encoded device such as a radiofrequency identification (RFID) chip, electronically encoded device, magnetically encoded device or optically encoded device. A unique identifier can be covalently or non-covalently attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

As used herein, the term “vessel” refers to an enclosure that contains a substance. The enclosure can be permanent or temporary with respect to the timeframe of a method set forth herein or with respect to one or more steps of a method set forth herein. Exemplary vessels include, but are not limited to, a well (e.g. in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel.

The embodiments set forth below and recited in the claims can be understood in view of the above definitions.

The present disclosure provides compositions that comprise one or more selected polypeptides having one or more desired or selected epitopes for affinity reagents. In some cases, the compositions described herein may comprise a plurality of selected polypeptides configured as a set of standard polypeptides. The standard polypeptide(s) may be selected from a variety of potential amino acid sequences that include the desired composition and number of epitopes in any given test polypeptide or set of test polypeptides. Standard polypeptides can include, for example, artificial or synthetic sequences, (e.g. sequences generated in silico or de novo), naturally derived sequences, (e.g. segments of known or naturally occurring polypeptide sequences), or combinations of these. As such, the desired epitopes can occur within one or more standard polypeptide and within a desired structural context provided by the chemical composition of the polypeptide. The lengths of the standard polypeptides described herein may vary in the number of amino acids as described herein for polypeptides, depending upon the desired structural characteristics for the selected polypeptide, including for example, the desired number of selected epitopes to be included, the spacing between epitopes, and the secondary or tertiary structural characteristics desired to be displayed by the epitopes within the polypeptides.

In some configurations, a set of polypeptides (e.g. standard polypeptides) can be non-naturally occurring. The set can be considered non-naturally occurring, for example, due to the set containing at least one polypeptide having a non-naturally occurring amino acid sequence. In some cases, all of the polypeptides in the set have non-naturally occurring amino acid sequences. However, presence of non-naturally occurring amino acid sequences is not necessarily required for a set of polypeptides (e.g. standard polypeptides) to be non-naturally occurring. For example, a set of polypeptides (e.g. standard polypeptides) can be non-naturally occurring by virtue of containing at least two amino acid sequences that are naturally occurring but do not naturally occur together in a natural setting. For example, a set of polypeptides can include polypeptides that do not co-occur in the same subcellular compartment, the same type of subcellular compartment (e.g. nucleus, mitochondria, chloroplast, endoplasmic reticulum, membrane, lysosome, peroxisome, or Golgi apparatus), the same cell, the same cell type, the same tissue, the same tissue type, the same biological fluid, the same type of biological fluid (e.g. blood, sweat, tears, lymph, sputum, or urine), the same organism or the same species of organism. A setting that has not been manufactured or synthetically altered by human art, science or industry will be understood to be a natural setting.

It will be understood that embodiments set forth herein in the context of non-naturally occurring amino acid sequence or non-naturally occurring polypeptides are exemplary. Those embodiments can readily be modified to use amino acid sequences or polypeptides that are naturally occurring in some settings but not in others. For example, an embodiment set forth herein in the context of using a non-naturally occurring amino acid sequence can be modified to use an amino acid sequence that is not native to an organism set forth herein even if the amino acid sequence is native to another organism. Generally, when using standard polypeptides to evaluate test proteins from a particular organism, it is advantageous to use standard polypeptides (or amino acid sequences thereof) that are non-native to that particular organism. Amino acid sequences can be compared using methods known in the art and using sequences having an appropriate length for comparison, such as a length exemplified herein for test polypeptides or standard polypeptides.

Optionally, a set of different polypeptides (e.g. standard polypeptides) can include at least one polypeptide having a non-naturally occurring amino acid sequence, wherein a set of different epitopes occurs in the set of different polypeptides, whereby the set of different polypeptides is a non-naturally occurring set of polypeptides. In a first configuration, individual polypeptides of the set of different polypeptides can each include a non-naturally occurring amino acid sequence, wherein a set of different epitopes occurs in the set of different polypeptides, each of the different epitopes occurring in the non-naturally occurring amino acid sequence of a subset of the different polypeptides, and the non-naturally occurring amino acid sequence of each of the different polypeptides including a plurality of different epitopes of the set of epitopes. However, not all polypeptides in a set of different polypeptides need necessarily nave non-naturally occurring amino acid sequences. In a second configuration, a subset of one or more individual polypeptides of the set of different polypeptides can each include a non-naturally occurring amino acid sequence, wherein a set of different epitopes occurs in the set of different polypeptides, each of the different epitopes occurring in the different polypeptides, and each of the different polypeptides including a plurality of different epitopes of the set of epitopes. As an option for the second configuration, individual polypeptides having naturally occurring or non-naturally occurring amino acid sequences can each include one or more different epitopes of a set of different epitopes. Each of the different polypeptides in a set of polypeptides set forth above can have a different combination of epitopes from the set of epitopes.

A set of epitopes can be configured for any of a variety of uses. For example, a set of epitopes can be configured to identify or characterize binding behavior of one or more affinity reagents. As such, one or more polypeptides that include epitopes from the set can be used as target polypeptides in a screen of candidate affinity reagents or as standard polypeptides in an assay for evaluating binding properties of an affinity reagent (e.g. binding strength, binding specificity or binding probability). Another use for one or more polypeptides that include epitopes from a set of epitopes is to serve as capture agent(s) (e.g. bait) for separating affinity reagents of interest from a sample. In another example, a set of epitopes can be configured to identify or characterize one or more test polypeptides based on binding to one or more known affinity reagents. As such, one or more polypeptides that include epitopes from the set can be used as standards or controls in an assay that utilizes one or more affinity reagents having known affinity for the epitopes. Binding of affinity reagents to standard polypeptides can be compared to binding of the affinity reagents to test polypeptides in order to identify or characterize the test polypeptides.

A set of epitopes can include individual epitopes that each have a particular amino acid composition. An epitope can include at least 1, 2, 3, 4, 5, 6 or more amino acids. Typically, the amino acids can be present as a contiguous sequence. For example, a set of epitopes can include dimers (sequences of 2 contiguous amino acids), trimers (sequences of 3 contiguous amino acids), tetramers (sequence of 4 contiguous amino acids), or pentamers (sequence of 5 contiguous amino acids). Optionally, a set of epitopes can include sequences in a particular size range such as at least 2, 3, 4, 5 or 6 contiguous amino acids. Alternatively or additionally, a set of epitopes can include sequences that include at most 6, 5, 4, 3 or 2 contiguous amino acids. Assuming random epitope sequences, shorter epitopes can be expected to occur in a larger number and variety of polypeptides in a given proteome compared to longer epitopes. An affinity reagent that recognizes shorter epitopes will generally be more promiscuous with regard to the variety of polypeptides it will bind in a given proteome sample. This can be beneficial for particular assays such as those set forth in in U.S. Pat. Nos. 10,473,654 or 11,282,585; US Pat App. Pub. No. 2023/0114905 A1; or Egertson et al., BioRxiv (2021), DOI: 10.1101/2021.10.11.463967, each of which is incorporated herein by reference. Typically, there is an inverse relationship between epitope length and promiscuity. Thus, longer epitopes can be useful when a lower level of promiscuity is desired.

In some cases, amino acids that define an epitope can be non-contiguous in the primary structure of a polypeptide. The amino acids may nevertheless be sufficiently proximal to each other in the secondary, tertiary, or quaternary structure of the polypeptide such that the amino acids can simultaneously interact with the binding pocket of an affinity reagent. This proximity can occur in the polypeptide when it is in its native state. In some cases, the proximity can occur when the protein is in a denatured state or in a misfolded state. Optionally, the proximity may also be achieved for the polypeptide in at least some of the conformations it achieves in a molten globule state. As such, an affinity reagent can interact with non-contiguous amino acids of a polypeptide when the polypeptide is in a native conformation, denatured state, misfolded state, or molten globule state.

An epitope that is non-contiguous can include two specific amino acid positions that are separated by a gap of one or more generic amino acid positions. The epitope can have the formula X₁αX₂, wherein X₁ and X₂ are individual amino acid positions occupied by a constant amino acid species, and α is a gap including one or more amino acid positions occupied by variable amino acid species. This configuration is illustrated by the epitope FXY in which the amino terminal phenylalanine (F) is separated from the carboxyterminal tyrosine (Y) by a position that can be occupied by any amino acid (X). Similarly, an epitope can have the formula X₁X₂αX₃ or X₁αX₂X₃, wherein X₃ is an individual amino acid position occupied by a constant amino acid species. An epitope can have more than one gap. For example, an epitope can include three constant amino acid positions and two gaps, wherein each of the gaps includes one or more variable amino acid positions. More specifically, an epitope can satisfy the formula X₁αX₂βX₃, wherein X₁, X₂ and X₃ are amino acid positions occupied by a constant amino acid species, and a and β are gaps, each gap including one or more amino acid positions occupied by variable amino acid species. By way of further example, an epitope having 2 constant amino acids can include a single gap; an epitope having 3 constant amino acid positions can include a gap between the first and second constant amino acids and/or a gap between the second and third constant amino acids; an epitope having 4 constant amino acid positions can include a gap between the first and second constant amino acids, a gap between the second and third constant amino acids and/or a gap between the third and fourth constant amino acids; and an epitope having 5 constant amino acid positions can include a gap between the first and second constant amino acids, a gap between the second and third constant amino acids, a gap between the third and fourth constant amino acids and/or a gap between the fourth and fifth constant amino acids. A gap that separates constant amino acid positions can include at least 1, 2, 3, 4, 5, 6 or more variable amino acid positions. Alternatively or additionally, the gap can include at most 6, 5, 4, 3, 2, or 1 variable amino acid positions. The size of the gap can be based on the nature of interactions between the epitope and an affinity reagent of interest. For example, in situations where the conformation of an epitope presents non-contiguous amino acids for binding to a particular affinity reagent, the number of intervening amino acid positions in the epitope that do not interact with the affinity reagent can be treated as a gap.

Optionally, a set of epitopes can be configured to omit one or more type of amino acid. The types of amino acids that can be omitted include, for example, one or more of A, R, N, D, C, Q, E, G, H, I, L, K, M, F, P, S, T, W, Y or V amino acids. For example, a set of epitopes can exclude amino acids having aliphatic R groups (e.g. G, A, V, L, I or P), polar neutral R groups (e.g. S or T), amide-containing R groups (e.g. N or Q), sulfur-containing R groups (e.g. M or C), aromatic R groups (e.g. F, Y or W), charged R groups (e.g. D, E, H, K, or R), anionic R groups (e.g. D or E), or cationic R groups (e.g. H, K or R). In some cases, a set of epitopes can be configured to exclude a type of amino acid that is known or suspected of being modified in a particular assay or other process that will employ the epitopes. For example, a set of epitopes can omit lysine (K) or Cysteine (C) amino acids due to these amino acids being modified in an assay or process, for example, to attach polypeptides of interest to a solid support. In another example, a set of epitopes can omit amino acids that are known or suspected of being post-translationally modified such as one or more of D, E, K, H, R, S, T, Y, N, Q or C. It will be understood that in some configurations a set of epitopes can include one or more types of amino acids selected from the above types of amino acids.

Optionally, a polypeptide can have a secondary structure that positions amino acids of an epitope to interact with a particular affinity reagent. For example, an epitope can be present in an alpha helix whereby the side chains of adjacent amino acid positions are offset along the peptide backbone by about 120°. As such, three side chains occur per turn of the alpha helix. In contrast, an epitope can be present in a beta strand whereby the side chains of adjacent amino acid positions have an angular offset of about 180°. As such, three adjacent side chains occur in 1.5 turns of the beta strand. The angles are approximate within a range that is determinable from a Ramachandran plot. Other secondary structures are possible such as those known to occur in loops and turns of polypeptide structures. A polypeptide can be designed to present amino acids of an epitope in a desired conformation by choice of amino acid content for the epitope as well as for the flanking regions of the epitope and in accordance with a secondary structure prediction algorithm. Empirical methods can also be used for polypeptide design.

In some cases, a set of epitopes can include amino acid sequences based on their prominence in a particular biological system such as the proteome of a particular organism or a collection of proteomes present in a particular environment, ecosystem or other population of organisms. For example, a set of epitopes of a given amino acid sequence length can include amino acid sequences in the top 1%, 2%, 3%, 4%, 5%, 10%, 15%, 20%, or 25% of all amino acid sequences that are of that length and encoded by a particular genome (or encoded by a particular combination of genomes). Optionally, a set of epitopes of a given amino acid sequence length can exclude amino acid sequences that occur in the bottom 75%, 80%, 85%, 90%, 95%, 96%, 97%, 98% or 99% of all amino acid sequences that are of that length and encoded by a particular genome (or encoded by a particular combination of genomes). Looking to the example of a set of trimer epitopes, the full set of possible trimers, given the 20 possible amino acid types, is 8000 trimers (i.e. 20³ trimers). A set of trimer epitopes can include epitopes selected from at least the most prominent 100, 200, 300, 500, 1×10³, or more amino acid trimers encoded by a particular genome (or encoded by a particular combination of genomes). Optionally, a set of trimer epitopes can exclude epitopes selected from at least the least prominent 100, 500, 1×10³, 3×10³, 5×10³, 7×10³ or more amino acid trimers encoded by a particular genome (or encoded by a particular combination of genomes). In this context, prominence is a measure of the distribution of epitope sequences in the polypeptides encoded by a given genome (or combination of genomes) independent of any differences in the expression levels for the polypeptides.

Exemplary organisms from which a set of epitopes can be selected include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. A polypeptide can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. A non-naturally occurring amino acid sequence can be non-native or otherwise absent from one or more of the above organisms.

A set of epitopes, such as those generated based on one or more of the criteria set forth herein, can be present in a polypeptide (e.g. standard polypeptide) or set of different polypeptides (e.g. set of different standard polypeptide). As such, one or more polypeptides can be designed to accommodate a particular set of epitopes. Characteristics of a set of different polypeptides that can be varied to accommodate a particular set of epitopes include, for example, the length (i.e. number of amino acids) of the polypeptides, the number of different polypeptides in the set, the number of epitopes present in each polypeptide, or the number of times each epitope occurs in a polypeptide of the set polypeptides. Optionally, a set of polypeptides can be a non-naturally occurring set of polypeptides, for example, by virtue of including at least one polypeptide having a non-naturally occurring amino acid sequence. Thus, a non-naturally occurring set of polypeptides can in some configurations include at least one naturally occurring polypeptide or naturally occurring amino acid sequence. In some cases, a set of polypeptides can be non-naturally occurring by virtue of combining two or more polypeptides that are not coincident in a naturally occurring organism or natural environment. Thus, all polypeptides in a non-naturally occurring set of polypeptides can be naturally occurring or can include naturally occurring amino acid sequences so long as the set, as a whole, is not naturally occurring.

In some configurations, a set of polypeptides (e.g. standard polypeptides) can include at least 2 different polypeptides, each of the different polypeptides including a sequence of at least 6 amino acids, wherein the sequence of at least 6 amino acids in each polypeptide of the different polypeptides is non-naturally occurring, wherein a set of epitopes occurs in the different polypeptides, the set of epitopes including at least 3 different epitopes, each of the epitopes including 3 contiguous amino acids, wherein each of the different epitopes in the set occurs in the sequence of at least 6 amino acids for at least 2 of the different polypeptides, and wherein the sequence of at least 6 amino acids for each of the different polypeptides includes at least 2 different epitopes of the set.

A set of polypeptides (e.g. standard polypeptides) can include a number of different polypeptides that satisfies a particular use of the set. A set of polypeptides (e.g. standard polypeptides) of the present disclosure can include at least 2, 3, 4, 5, 10, 15, 20, 25, 30, 35, 40, 45, 50, 75, 100 or more different polypeptides. Alternatively or additionally, a set of polypeptides (e.g. standard polypeptides) can include at most 100, 75, 50, 45, 40, 35, 30, 25, 20, 15, 10, 5, 4, 3, or 2 different polypeptides. Generally, the different polypeptides differ with respect to their amino acid sequences. Looking to the example of a set of standard polypeptides used when assaying test polypeptides with affinity reagents, the set can include relatively few members when a relatively low number of affinity reagents is used or when the amino acid sequence diversity of the test polypeptides is low. As the number of affinity reagents is increased or as the sequence diversity of the test polypeptides increases, the number of different standard polypeptides in the set can be increased. For example, the number of different polypeptides in set of standard polypeptides can be at most 10%, 1%, 0.1%, 0.01%, or 0.001% of the number of different affinity reagents that recognize at least one epitope in the different polypeptides, or less.

A set of polypeptides (e.g. standard polypeptides) can include amino acid sequences having particular lengths. For example, the lengths for amino acid sequences in a set of different polypeptides (e.g. standard polypeptides) can be at least 2, 3, 4, 5, 6, 10, 15, 20, 25, 30, 40, 50, 60, 70, 80, 90, 100, 150, 200 or more amino acids. Alternatively or additionally, the lengths for amino acid sequences in a set of different polypeptides (e.g. standard polypeptides) can be at most 200, 150, 100, 90, 80, 70, 60, 50, 40, 30, 25, 20, 15, 10, 6, 5, 4, 3, or 2 amino acids. The aforementioned sequence lengths can refer to the full-length amino acid sequences of the polypeptides in the set or, alternatively, to a contiguous portion of the full-length amino acid sequences of the polypeptides in the set. Moreover, the aforementioned sequence lengths can refer to one, some or all polypeptides in a set of different polypeptides. Accordingly, all amino acid sequences in a set of polypeptides can be the same length. Alternatively, a set of polypeptides can include different length amino acid sequences. It will be understood that any polypeptide set forth herein, whether or not included in a set of standard polypeptides, can include an amino acid sequence of a length set forth above.

A polypeptide (e.g. standard polypeptide) can be characterized in terms of the number of epitopes present in its amino acid sequence. For example, a polypeptide (e.g. standard polypeptide) can include at least 1, 2, 3, 4, 5, 6, 7, 8, 9, 10, 12, 14, 16, 18, 20, 25 or more epitopes. Alternatively or additionally, a polypeptide (e.g. standard polypeptide) can include at most 25, 20, 18, 16, 14, 12, 10, 9, 8, 7, 6, 5, 4, 3, 2 or 1 epitopes. The epitopes can be selected from a set of epitopes such as a set of epitopes set forth herein. Each of the epitopes in a given polypeptide can be different from all other epitopes in that polypeptide. For example, the amino acid sequence of a given epitope in a polypeptide can differ from the amino acid sequences of all other epitopes in the polypeptide. Indeed, the amino acid sequence of all epitopes in a polypeptide can differ from the amino acid sequences of all other epitopes in the polypeptide. Alternatively, a polypeptide can include two or more epitopes having the same amino acid sequence, the two or more epitopes being located at different positions within the overall sequence of the polypeptide. In some cases, two or more epitopes can overlap. For example, a sequence of 4 amino acids (e.g. HHYH) contains two trimer epitopes (e.g. HHY and HYH). The two trimer epitopes, although having a partial overlap, are nonetheless located at different positions within the overall sequence of the polypeptide

A polypeptide (e.g. standard polypeptide) can include one or more amino acids that provide structural or functional characteristics other than serving as epitopes. For example, a polypeptide can include a spacer between two epitopes. The spacer can function to spatially separate the two epitopes in the sequence of the polypeptide and, optionally, can also facilitate a conformation for the polypeptide that positions one or both epitopes for improved binding to an affinity reagent (compared to absence of the spacer). Optionally, the spacer can include one or more amino acids that are relatively inert to binding an affinity reagent of interest. For example, a spacer can include a glycine or a sequence including 2, 3, 4 or more glycines. This can be beneficial since glycines are relatively non-antigenic for antibodies. In another example, a spacer can include an amino acids having an aliphatic R group (e.g. G, A, V, L, I or P) or a sequence of 2, 3, 4 or more amino acids having aliphatic R groups. This can be beneficial since aliphatic R groups are relatively non-aptagenic for aptamers having the standard four DNA bases. Non-peptide linkers can also be useful as spacers between epitopes of a polypeptide. A polypeptide can also include a sequence of amino acids that is known or suspected of forming a desired secondary, tertiary or quaternary structure. For example, sequences that form alpha helices, beta sheets, turns or other motifs can be useful.

Optionally, a set of polypeptides (e.g. standard polypeptides), or subset thereof, can share a structural or functional characteristic imparted by one or more amino acids. For example, a plurality of standard polypeptides can include a spacer exemplified above. In another example, a plurality of standard polypeptides can include a universal amino acid sequence. As such, a set of polypeptides (e.g. standard polypeptides), or subset thereof, can include a common primary structure, such as a sequence of at least 2, 3, 4, 5, 6, 7, 8, 9, 10 or more amino acids, even though individual polypeptides in the set differ with respect to the number or type of epitopes they contain. As another option, a set of polypeptides (e.g. standard polypeptides), or subset thereof, can include a common secondary, tertiary or quaternary structural motif. In some configurations, a set of polypeptides, or subset thereof, can share a common chemical property, such as having the same pKa, pI, solubility, net charge, net hydrophobicity, net hydrophilicity, net polarity, mass, length (i.e. number of amino acids), or the like. Optionally, a plurality of polypeptides can include a common scaffold or background structure that nonetheless accommodates epitopes that differ between individual polypeptides in the plurality.

A set of different polypeptides (e.g. standard polypeptides) can be characterized in terms of the minimum number of epitopes present per polypeptide. For example, a set of different polypeptides (e.g. standard polypeptides) can include at least 1, 2, 3, 4, 5, 6, 7, 8, 9, 10, 12, 14, 16, 18, 20, 25 or more epitopes per polypeptide. Alternatively or additionally, a set of polypeptides (e.g. standard polypeptides) can be characterized in terms of the maximum number of epitopes present per polypeptide. For example, a set of different polypeptides (e.g. standard polypeptides) can include at most 25, 20, 18, 16, 14, 12, 10, 9, 8, 7, 6, 5, 4, 3, 2 or 1 epitopes per polypeptide. The epitopes can be selected from a set of epitopes such as a set of epitopes for a given set of affinity reagents or a of set of epitopes set forth herein.

A set of different polypeptides (e.g. standard polypeptides) can be characterized in terms of the number of epitopes present in the set taken as a whole. For example, a set of different polypeptides (e.g. standard polypeptides) can include at least 2, 3, 4, 5, 10, 20, 25, 50, 75, 100, 150, 200, 250, 300, 400, 500, or more different epitopes. The epitopes can be selected from a set of epitopes such as a set of epitopes for a given set of affinity reagents or a of set of epitopes set forth herein.

Epitopes having a particular amino acid composition or sequence can be present in multiple different polypeptides in a set of polypeptides (e.g. standard polypeptides). As such, a given epitope can be present redundantly in a set of polypeptides. For example, a given epitope can occur in at least 2, 3, 4, 5, 6, 7, 8, 9, 10 or more different polypeptides (e.g. standard polypeptides) in a set. Alternatively or additionally, a given epitope can occur in at most 10, 9, 8, 7, 6, 5, 4, 3, or 2 different polypeptides (e.g. standard polypeptides) in a set. Optionally, a given epitope can be present in a subset of the different polypeptides in a set of polypeptides. For example, a given epitope that is present in multiple different polypeptides of a standard polypeptide set can also be absent in at least one standard polypeptide in the set. Accordingly, a given epitope that is present in multiple different polypeptides (e.g. standard polypeptides) of a set of polypeptides can be absent from at least 1, 2, 3, 4, 5, 6, 7, 8, 9, 10 or more of the different polypeptides in the set. Alternatively or additionally, a given epitope that is present in multiple different polypeptides of a set of polypeptides (e.g. standard polypeptide) can be absent from at most 10, 9, 8, 7, 6, 5, 4, 3, 2 or 1 of the different polypeptides in the set. Polypeptides that include a particular epitope can function, for example, as positive controls for an affinity reagent that recognizes the epitope. On the other hand, polypeptides that exclude a particular epitope can function, for example, as negative controls for an affinity reagent that recognizes the epitope.

The redundancy exemplified above for a given epitope can be extended to some or all epitopes in a set of epitopes. Accordingly, some or all epitopes in a given set of epitopes can each occur in at least 2, 3, 4, 5, 6, 7, 8, 9, 10 or more different polypeptides in a set of polypeptides (e.g. standard polypeptides). Alternatively or additionally, some or all epitopes in a given set can each occur in at most 10, 9, 8, 7, 6, 5, 4, 3, or 2 different polypeptides in a set of polypeptides (e.g. standard polypeptides). Moreover, some or all epitopes in a given set of epitopes that are present in multiple different polypeptides of a set of polypeptides (e.g. standard polypeptides) can be absent from at least 1, 2, 3, 4, 5, 6, 7, 8, 9, 10 or more of the different polypeptides in the set. Alternatively or additionally, some or all epitopes in a given set of epitopes that are present in multiple different polypeptides of a set of polypeptides (e.g. standard polypeptides) can be absent from at most 10, 9, 8, 7, 6, 5, 4, 3, 2 or 1 of the different polypeptides in the set.

One or more polypeptides (e.g. standard polypeptides or test polypeptides) that are included in a method or composition of the present disclosure can be soluble in aqueous solution. Such polypeptides are particularly useful for assays, screens or separation procedures carried out in aqueous solvent. For example, standard polypeptides can be selected for inclusion in a set of standard polypeptides based, at least in part, on their aqueous solubility. One or more different polypeptides, for example, present in a set of polypeptides, can have a predicted solubility of at least 0.3, 0.4, 0.5, 0.6, 0.7 or higher. Alternatively or additionally, one or more different polypeptides, for example, present in a set of polypeptides, can have a predicted solubility of at most 0.8, 0.7, 0.6, 0.5, 0.4, 0.3 or lower. Solubility can be scored using a known algorithm such as protein-sol (see Hebditch et al., 33: 3098-3100 Bioinformatics (2017), which is incorporated herein by reference). Aqueous solubility of polypeptides can be facilitated by including polar or charged amino acids in the polypeptides, for example, at solvent exposed regions of the molecules. It will be understood that one or more polypeptides can be configured for use in a non-aqueous environment such as a non-polar solvent, organic solvent, membrane or oil. Solubility of polypeptides in non-aqueous environments can be facilitated by including non-polar or non-charged amino acids in the polypeptides, for example, at solvent exposed regions of the molecules. As such, the polypeptide(s) can be selected for solubility in non-aqueous environments. Alternatively, a set of polypeptides can include member polypeptides having different solubility values. This can be useful, for example, to separate or distinguish one polypeptide from another in a method set forth herein.

One or more polypeptides that are included in a method or composition of the present disclosure can have an isoelectric point (pI) in a particular range of values. One or more different polypeptides (e.g. standard polypeptides), for example, present in a set of polypeptides (e.g. standard polypeptides), can have a pI of at least 4.0, 5.0, 6.0, 7.0, 8.0, 9.0, 10.0, 11.0 12.0 or more. Alternatively or additionally, one or more different polypeptides (e.g. standard polypeptides), for example, present in a set of polypeptides (e.g. standard polypeptides), can have a pI of at most 12.0, 11.0, 10.0, 9.0, 8.0, 7.0, 6.0, 5.0, 4.0 or less. In some cases, different polypeptides (e.g. standard polypeptides) that are present in a set can have pI values that are substantially similar to each other. For example, the polypeptides (e.g. standard polypeptides) in a set can have pI values that vary by less than 3.0, 2.5, 2.0, 1.5, 1.0 or less. The preceding variance ranges can center around a given pI value such as 4.0, 5.0, 6.0, 7.0, 8.0, 9.0, 10.0, 11.0 or 12.0. Alternatively, a set of polypeptides (e.g. standard polypeptides) can include member polypeptides having different pI values. This can be useful, for example, to separate or distinguish one standard polypeptide from another in a method set forth herein.

Other characteristics that can be similar for two or more polypeptides that are included in a method or composition of the present disclosure include, but are not limited to pKa, overall charge, pH dependent charge, pH dependent solubility, hydrophobicity, hydrophilicity, polarity, non-polarity, Stoke' s radius, secondary structure, tertiary structure, mass, amino acid sequence length, or charge-to-mass ratio. Similarity of characteristics can be selected to achieve a desired function for a set of polypeptides (e.g. standard polypeptides). For example, the polypeptides can have similar charge-to-mass ratio such that the polypeptides will co-migrate in an electrophoretic separation. Similarity in pH dependent charge or solubility can be useful for procedures in which the polypeptides will be exposed to a given pH or to changes in pH while being used in a method set forth herein or prepared for use. In some cases, it may be desirable for two or more polypeptides to differ with respect to one or more characteristics. Characteristics that can differ for polypeptides included in a method or composition set forth herein include, but are not limited to solubility, pI, pKa, overall charge, pH dependent charge, pH dependent solubility, hydrophobicity, hydrophilicity, polarity, non-polarity, Stoke's radius, secondary structure, tertiary structure, mass, amino acid sequence length, or charge-to-mass ratio. The differences can be useful for separating or distinguishing one polypeptide from one or more other polypeptides in a set of polypeptides. For example, a standard polypeptide can have a unique charge-to-mass ratio such that it can be separated from other standard polypeptides in an electrophoretic separation. Similarity or differences in secondary or tertiary structure can be identified, for example, using an algorithm such as PSIPRED (Jones J. Mol. Biol. 292: 195-202(1999), and Buchan et al. Nucl. Acids Res. https://doi.org/10.1093/nar/gkz297 (2019), each of which is incorporated herein by reference) or DSSP (Wouter et al., Nucl. Acids Res. 43: D364-D368 (2015) and Kabsch et al., Biopolymers 22: 2577-2637 (1983), each of which is incorporated herein by reference).

Two or more polypeptides that are included in a method or composition of the present disclosure, can include a universal tag. Any of a variety of labels can be used as universal tags. The tags are referred to as being universal with respect to being common to multiple members in a given set of polypeptides. For example, all polypeptides in a set of standard polypeptides can have the same luminophore moiety such that detection of the luminophore moiety on an individual polypeptide indicates that the polypeptide is a member of a set of standard polypeptides that utilized the luminophore as a universal tag. A particularly useful universal tag is a universal amino acid sequence. For example, polypeptides in a set can include a region of amino acid sequence that is common to the polypeptides in the set. Of course, the polypeptides of the set can differ from each other overall due to having regions of sequence that differ between the polypeptides. In some cases, a universal amino acid sequence can include one or more epitopes such as an epitope set forth herein.

One or more standard polypeptides can have amino acid sequences that differ from the amino acid sequences that are known or suspected of being present in a particular biological system. The biological system can be an organism, collection of organisms, ecosystem, environmental sample, forensic sample, biopsy or the like. In some cases, the biological system is to be manipulated or detected in a method set forth herein. As such, a standard polypeptide or set of standard polypeptides can lack amino acid sequences found in a collection of test polypeptides that is to be manipulated or detected in a method set forth herein.

A standard polypeptide that is to be used in combination with a plurality of test polypeptides can be configured to have a combination of epitopes that is distinguishable from the combination of epitopes present in any of the test polypeptides in the plurality. Thus, the standard polypeptide can be distinguished from the test polypeptides using an appropriate combination of affinity reagents. Accordingly, the combination of epitopes found in a standard polypeptide can be unique when compared to all individual polypeptides in a particular collection of test polypeptides. The collection can include, for example, all naturally occurring amino acid sequences, all native amino acid sequences found in one or more organisms (e.g. one or more organism set forth herein), all native amino acid sequences expressed in a particular cell type or tissue type, or all naturally occurring amino acid sequences in a particular ecosystem. A combination of epitopes found in a standard polypeptide can be unique when compared to a portion of a proteome including, for example, a portion that is found in a subcellular component such as an organelle, membrane or cytosol, whether or not the portion is absent from another subcellular component. A combination of epitopes found in a standard polypeptide can be unique when compared to a portion of a proteome that is obtained by fractionating a biological sample, such as a soluble fraction that substantially lacks membrane proteins, a membrane fraction that substantially lacks soluble proteins, a chromatographic fraction, a precipitate from an affinity extraction, or the like.

A standard polypeptide can be designed to have a combination of epitopes that falls outside of a radius of epitope combinations found in a cluster of test polypeptides such as those set forth above or set forth elsewhere herein. Given epitope combinations for a set of polypeptides a distance metric between polypeptides can be defined as the number of changes of presence/absence of epitopes in the epitope set. For instance, the epitope combinations for a set of 3 polypeptides probed with 4 unique affinity reagents can be {1, 0, 0, 1 }, {0, 0, 0, 1 }, and {1, 1, 1, 1} where 1 denotes presence of binding and 0 denote absence of binding. The distance, as defined above, between polypeptides 1 and 2 would be 1 as there is only 1 position in which they differ. A “radius” can be set at 1 and applied to the second polypeptide in the set to generate a non-naturally occurring polypeptide (assuming the set of three polypeptides is the universe) with the epitope combination {0, 1, 0, 1}. This distance is limited by the number of affinity reagents used to probe the polypeptides. Smaller distances correspond to more similar sequences and can be used as a decoy for purposes of identifying polypeptides.

The present disclosure provides a polypeptide (e.g. standard polypeptide) having the amino acid sequence of any one of SEQ ID NOs: 1 to 40. Also provided in a set of polypeptides (e.g. standard polypeptides) including at least two amino acid sequences selected from SEQ ID NOs: 1 to 40. The sequences can be selected from one or more of Tables II, IV and VI, herein below. For example, a set of polypeptides (e.g. standard polypeptides) can include at least 2, 3, 4, 5, 6, 7, 8, 9, 10, 11, 12, 13, or 14 of the sequences set forth in Table II. Alternatively or additionally, a set of polypeptides (e.g. standard polypeptides) can include at least 2, 3, 4, 5, 6, 7, 8, 9, 10, 11, 12, 13, 14, 15 or 16 of the sequences set forth in Table IV. Optionally, a set of polypeptides (e.g. standard polypeptides) can include at least 2, 3, 4, 5, 6, 7, 8, 9, 10, or 11 of the sequences set forth in Table VI. It will be understood that in some cases, one or more of the sequences listed in Tables II, IV or VI can be absent in a set of polypeptides (e.g. standard polypeptides).

A sequence set forth in SEQ ID NOs: 1 to 40 can constitute at least a portion of the amino acid sequence of a standard polypeptide. In some cases, a sequence set forth in SEQ ID NOs: 1 to 40 constitutes the full sequence of a standard polypeptide. Moreover, a standard polypeptide can include two or more sequence regions such that two or more sequences set forth in SEQ ID NOs: 1 to 40 are present in a single polypeptide molecule.

A polypeptide (e.g. a standard polypeptide or test polypeptide) can be modified for any of a variety of uses. For example, a polypeptide can be modified to facilitate attachment to a moiety, substance or object. A polypeptide can be modified at a reactive moiety such as (i) an amine that is present at the amino terminus of the polypeptide or in the side chain of a lysine, histidine or arginine side chain; (ii) a sulfur that is present in the side chain of a cysteine or methionine; (iii) a carboxylate that is present at the carboxy terminus of a polypeptide or in the side chain of an aspartic acid or glutamic acid; (iv) an oxygen that is present in the side chain of a serine, threonine or tyrosine; or (v) an amide that is present in the side chain of a glutamine or asparagine. Modifications known in the art of chemical biology and biochemistry can be used including, for example, those available from commercial suppliers such as ThermoFisher, Waltham MA or Sigma Aldrich, St. Louis, MO. Other useful chemistries are set forth in U.S. Pat. No. 11,203,612, US Pat. App. Pub. Nos. 2022/0162684 A1 or 2022/0290130 A1, each of which is incorporated herein by reference.

A polypeptide (e.g. a standard polypeptide or test polypeptide) can be attached to a moiety, substance or object. Exemplary attachments include, but are not limited to, covalent or non-covalent attachments such as those set forth in US Pat. App. Pub. Nos. 2021/0101930 A1 or 2022/0290130 A1, each of which is incorporated herein by reference. For example, a polypeptide can be attached to a moiety, substance or object via non-covalent interactions between a receptor and ligand. Exemplary receptor-ligand pairs that can be used include, but are not limited to, an antibody, such as a full-length antibody or functional fragment thereof which binds to an epitope; (strept)avidin (or analogs thereof) which binds to biotin (or analogs thereof); complementary nucleic acids which bind each other; nucleic acid aptamers and their ligands; lectins and carbohydrates; or the like. A large variety of covalent chemistries are available for attaching polypeptides to moieties, substances or objects. Click chemistry can be particularly useful. For example, attachment can be accomplished by chemical reaction of a click moiety on a moiety, substance or object with a reactive moiety on a polypeptide. The chemical conjugation may proceed via an amide formation reaction, reductive amination reaction, N-terminal modification, thiol Michael addition reaction, disulfide formation reaction, copper(I)-catalyzed alkyne-azide cycloaddition (CuAAC) reaction, strain-promoted alkyne-azide cycloaddtion reaction (SPAAC), Strain-promoted alkyne-nitrone cycloaddition (SPANC), inverse electron-demand Diels-Alder (IEDDA) reaction, oxime/hydrazone formation reaction, free-radical polymerization reaction, or a combination thereof. A polypeptide can be attached to a moiety, substance or object via a SpyTag/SpyCatcher system (See, Zakeri et al. Proceedings Nat'l Acad. Sciences USA. 109 (12): E690-7 (2012); U.S. Pat. Nos. 9,547,003 or 11,059,867 or US Pat. App. Pub. No. 2022/0135628 A1, each of which is incorporated herein by reference). In this system, a 13 amino acid tag polypeptide (Spy Tag) forms a first coupling handle, with a 12.3 kDa polypeptide (Spy-Catcher) forming the partner to the first coupling handle. Optionally, the Spy Catcher can be attached to a polypeptide. The Spy Catcher can irreversibly bond to a Spy Tag on a moiety, substance or object through an isopeptide bond. As will be appreciated, either the Spy Tag or the Spy Catcher can be on the moiety, substance or object, and a polypeptide can be functionalized with the other partner. Exemplary moieties, substances and objects to which polypeptides can be attached include, but are not limited to, particles, solid supports, array addresses and labels such as those set forth in further detail herein.

A polypeptide (e.g. a standard polypeptide or test polypeptide) can include a post-translational modification (PTM) moiety. The PTM moiety can be added by a biological system, by one or more components of a biological system or by a synthetic procedure. In some configurations, a standard polypeptide can include a site that is modifiable to generate a post-translational modification. A PTM moiety may be present at the site or absent from the site to suit a particular use of the polypeptide. The site can include an amino acid of a type that is prone to post-translational modification and in some cases can include a sequence of amino acids that is recognized by, or otherwise facilitates, modification by an enzyme or other biochemical agent. Exemplary PTM moieties include, but are not limited to, myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization.

A post-translational modification may occur at a particular type of amino acid residue in a polypeptide. Optionally, the amino acid residue can be located in an epitope of a polypeptide (e.g. a standard polypeptide or test polypeptide). For example, a phosphoryl moiety can be present on a serine, threonine, tyrosine, histidine, cysteine, lysine, aspartate or glutamate residue. In another example, an acetyl moiety can be present on the N-terminus or on a lysine of a polypeptide. In another example, a serine or threonine residue of a polypeptide can have an O-linked glycosyl moiety, or an asparagine residue of a polypeptide can have an N-linked glycosyl moiety. In another example, a proline, lysine, asparagine, aspartate or histidine amino acid of a polypeptide can be hydroxylated. In another example, a polypeptide can be methylated at an arginine or lysine amino acid. In another example, a polypeptide can be ubiquitinated at the N-terminal methionine or at a lysine amino acid. It will be understood that one or more polypeptides of the present disclosure can be devoid of one or more of the PTM moieties set forth herein. A method of the present disclosure can include a step of modifying one or more polypeptide (e.g. standard polypeptide), for example, by adding a PTM moiety or removing a PTM moiety.

One or more polypeptides (e.g. a standard polypeptide or test polypeptide) can include a label. For example, an exogenous label can be attached to a polypeptide. The attachment can be covalent or non-covalent. Different standard polypeptides in a set of standard polypeptides can include the same label as each other (e.g. universal label) or they can be distinguished from each other by different labels. Exemplary labels include, without limitation, a fluorophore, luminophore, chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like. A label can produce any of a variety of detectable signals including, for example, an optical signal such as absorbance of radiation, luminescence (e.g. fluorescence or phosphorescence) emission, luminescence lifetime, luminescence polarization, or the like; Rayleigh and/or Mie scattering; magnetic properties; electrical properties; charge; mass; radioactivity or the like. A label may produce a signal with a characteristic frequency, intensity, polarity, duration, wavelength, sequence, or fingerprint. A label need not directly produce a signal. For example, a label can bind to a receptor or ligand having a moiety that produces a characteristic signal. Such labels can include, for example, nucleic acids that are encoded with a particular nucleotide sequence, avidin, biotin, non-peptide ligands of known receptors, or the like.

One or more polypeptides (e.g. a standard polypeptide or test polypeptide) can be attached to one or more particles. For example, each particle can be attached to a single polypeptide molecule. As such, each particle can be attached to one and only one polypeptide. In some configurations, each particle can be attached to a plurality of polypeptides. The polypeptides that are attached to a given particle can have different amino acid sequences from each other. For example, a plurality of polypeptides that is attached to a particle can include two or more amino acid sequences, such as two or more of the sequences set forth in SEQ ID Nos: 1 to 40. In other cases, polypeptides that are attached to a given particle can have the same sequences as each other. For example, a plurality of polypeptides that is attached to a particle can share a common amino acid sequence, such as a sequence set forth in any one of SEQ ID Nos: 1 to 40.

Structured nucleic acid particles are particularly useful, such as those that include nucleic acid origami. A nucleic acid origami can include one or more nucleic acids having a variety of overall shapes such as a disk, tile, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. In some configurations, a nucleic acid origami may include a scaffold nucleic acid and a plurality of staple nucleic acids. The scaffold can be configured as a single, continuous strand of nucleic acid, and the staples can be formed by nucleic acids that hybridize, at least in part, with the scaffold nucleic acid. A structured nucleic acid particle may include regions of single-stranded nucleic acid, regions of double-stranded nucleic acid, or combinations thereof.

In some configurations, a nucleic acid origami includes a scaffold composed of a nucleic acid strand hybridized to a plurality of oligonucleotides. A scaffold strand can be linear (i.e. having a 3′ end and 5′ end) or circular (i.e. closed such that the scaffold lacks a 3′ end and 5′ end). A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 5000 or more nucleotides. Alternatively or additionally, a scaffold strand may include at most about 5000, 1000, 500, 100 or fewer nucleotides.

A plurality of oligonucleotides that is hybridized to a scaffold strand can include at least 2, 5, 10, 50, 100 or more oligonucleotides. A first region of an oligonucleotide sequence can be hybridized to a scaffold strand while a second region of the oligonucleotide is not hybridized to the scaffold strand. One or both of the regions can be located at or near an end of the oligonucleotide (e.g. the 5′0 end or the 3′ end), or in a region that is between the end regions of the oligonucleotide. The second region can be in a single stranded state or, alternatively, can participate in a hairpin or other self-annealed structure in the oligonucleotide. Optionally, the second region of the oligonucleotide can form a covalent or non-covalent bond with a polypeptide. An oligonucleotide that is included in a nucleic acid origami can have a length of at least about 10, 25, 50, 100 or more nucleotides. Alternatively or additionally, an oligonucleotide may have a length of no more than about 100, 50, 25, 10, or fewer nucleotides.

Two or more sequence regions of an oligonucleotide can be hybridized to a scaffold strand, for example, to function as a ‘staple’ that restrains the structure of the scaffold. For example, a single oligonucleotide can hybridize to two regions of a scaffold that are separated from each other in the primary sequence of the scaffold. As such, the oligonucleotide can function to retain those two regions of the scaffold in proximity to each other or to otherwise constrain the scaffold to a desired conformation. One or both of the hybridized regions of a staple can be located at or near an end of the oligonucleotide (e.g. the 5′0 end or the 3′ end), or in a region of the oligonucleotide that is between the end regions. Two sequence regions of an oligonucleotide staple that hybridize to a scaffold can be adjacent to each other in the oligonucleotide sequence or separated by a spacer region that does not hybridize to the scaffold.

A polypeptide (e.g. a standard polypeptide or test polypeptide) can be attached to nucleic acid origami via a scaffold component or oligonucleotide component of the origami structure. For example, the scaffold or oligonucleotide can include one or more nucleotide analog(s) that attach covalently or non-covalently to a polypeptide. Further examples of structured nucleic acid particles are set forth, for example, in U.S. Pat. No. 11,203,612; US Pat. App. Pub. Nos. 2022/0162684 A1 or 2022/0290130 A1, each of which is incorporated herein by reference.

A particle need not be composed primarily of nucleic acid and, in some cases, may be devoid of nucleic acids. For example, a particle can be composed of a solid support material. Whatever the composition, a particle may have any of a variety of sizes and shapes to accommodate use in a desired application. For example, a particle can have a regular or symmetric shape or, alternatively, a particle can have an irregular or asymmetric shape. The shape can be rigid or pliable. Optionally, a particle can have a minimum, maximum or average length of at least about 50 nm, 100 nm, 500 nm, 1 mm, or more. Alternatively or additionally, a particle can have a minimum, maximum or average length of no more than about 1 mm, 500 nm, 100 nm, 50 nm, or less. A particle can be characterized with respect to its footprint (e.g. occupied area on a surface). Optionally, the minimum, maximum or average area for a particle footprint can be at least about 10 nm², 100 nm², 1 μm², 10 μm², 100 μm², 1 mm² or more. Alternatively or additionally, the minimum, maximum or average area for a particle footprint can be at most about 1 mm², 100 μm², 10 μm², 1 μm², 100 nm², 10 nm², or less.

One or more polypeptides (e.g. a standard polypeptide or test polypeptide) can be in fluid-phase, such as an aqueous liquid. Alternatively, one or more polypeptides can be immobilized, for example, being attached to a solid support. In particular configurations of the method set forth herein, one or more polypeptides can be in fluid-phase for some steps and immobilized on a solid support for other steps. For example, one or more polypeptides can be in fluid-phase when delivered to a solid support and one, some or all of the polypeptides can then be attached to a solid support, thereby becoming immobilized.

A solid support can be configured in any of a variety of ways. Solid supports that are configured as particles can be particularly useful, for example, as set forth above. A plurality of polypeptides (e.g. a standard polypeptide or test polypeptide) can be attached to a plurality of particles. The plurality can include, for example, at least 2, 5, 10, 100, 1×10³, 1×10⁶, 1×10⁹ or more particles. Some or all of the particles in the plurality can be attached to a polypeptide having an amino acid sequence set forth herein. Individual polypeptides of a set of polypeptides can each be attached to a respective particle of a plurality of particles. For example, individual particles can each be attached to a single amino acid sequence of SEQ ID NOs: 1 to 40. Optionally, individual particles can each be attached to a single polypeptide having an amino acid sequence of SEQ ID NOs: 1 to 40. Optionally, a plurality of particles can include standard polypeptides (e.g. polypeptides having amino acid sequence(s) set forth in one or more of SEQ ID Nos: 1 to 40) and test polypeptides (e.g. polypeptides having one or more sequences encoded by an organism).

Another useful configuration for a solid support is as an array having a plurality of addresses. Optionally, individual addresses in an array can each be attached to a single polypeptide molecule. As such, an address can be attached to one and only one polypeptide. In some configurations, individual addresses can each be attached to a plurality of polypeptides. Multiple polypeptides that are attached to a given address can have different amino acid sequences from each other. For example, a plurality of polypeptides that is attached to an address can include two or more amino acid sequences, such as two or more of the sequences set forth in SEQ ID Nos: 1 to 40. In other cases, multiple polypeptides that are attached to a given address can have the same sequences as each other. For example, a plurality of polypeptides that is attached to an address can share a common amino acid sequence, such as a sequence set forth in any one of SEQ ID Nos: 1 to 40.

An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10³, 1×10⁶, 1×10⁹, 1×10¹², or more addresses. Alternatively or additionally, an array can include at most 1×10¹², 1×10⁹, 1×10⁶, 1×10³ or fewer addresses. Some or all addresses in an array can be attached to a polypeptide having an amino acid sequence set forth herein. Individual polypeptides of a set of polypeptides can each be attached to a respective address of an array. For example, individual addresses of an array can each be attached to a single amino acid sequence of SEQ ID NOs: 1 to 40. Optionally, individual addresses of an array can each be attached to a single polypeptide having an amino acid sequence of SEQ ID NOs: 1 to 40. Optionally, an array can include one or more addresses attached to standard polypeptides (e.g. polypeptides having amino acid sequence(s) set forth in one or more of SEQ ID Nos: 1 to 40) and one or more addresses attached to test polypeptides (e.g. polypeptides having one or more sequences encoded by an organism).

In some cases, a polypeptide (e.g. a standard polypeptide or test polypeptide) can be attached to a solid support surface via a particle. The particle can be composed of solid support material or other materials such as nucleic acid (e.g. structured nucleic acid particle). A particle can be attached to a surface via covalent or non-covalent means such as those set forth herein in the context of attaching polypeptides to nucleic acids or solid supports. Individual addresses of an array can each include a single particle. As such individual addresses can each include one and only one particle. Alternatively, individual addresses in an array can each be attached to a plurality of particles.

Whether in fluid-phase or immobilized on a solid support, one or more polypeptides (e.g. a standard polypeptide or test polypeptide) can be present in a vessel such as a flow cell. A flow cell can be particularly useful for manipulating or detecting polypeptides. A flow cell can include a detection region such as a region that is visible via an optically transparent window. The detection region can be fluidically accessible from outside the flow cell. For example, the flow cell can include an ingress through which fluid can be introduced to the detection region and an egress through which fluid can be evacuated from the detection region. Polypeptides can optionally be immobilized at the detection region, for example, via attachment to an array.

One or more polypeptides (e.g. a standard polypeptide or test polypeptide) can be present in a detection apparatus. For example, the polypeptide(s) can be present in a vessel, such as a flow cell, and the vessel can be engaged with the detection apparatus. The vessel can be permanently or temporarily engaged with a detection apparatus. A detection apparatus can be configured to detect contents of a vessel, for example, by acquiring signals arising from the vessel. For example, a detection apparatus can be configured to acquire optical signals through an optically transparent window of a flow cell. Optionally, the detection apparatus can be configured for luminescence detection, for example, having an optical train that delivers radiation from an excitation source (e.g. a laser or lamp) and through a window of the vessel to one or more polypeptides in the vessel. The detection apparatus can further include a camera or other detector that acquires signals transmitted through the window of the vessel and through an optical train. Optionally excitation and emission can be transmitted through the same optical train; however, separate optical trains can also be useful.

A detection apparatus can include a fluidic system. Optionally, the fluidic system can be configured for fluidic communication with a vessel. One or more steps of a method set forth herein can occur in the vessel. In some configurations, a fluidic system of a detection apparatus can include one or more reservoirs containing assay components set forth herein such as at least one affinity reagent(s) or polypeptide(s) set forth herein. Affinity reagents that are present in a detection apparatus, for example in a reservoir, can be configured to recognize one or more epitopes in a set of epitopes or set of standard polypeptides set forth herein. A fluidic system of a detection apparatus set forth herein can be configured to transfer assay components from one or more reservoirs to a vessel. One or more reactions occurring in the vessel can be detected by the detection apparatus, for example, be acquiring signals resulting from the reaction(s). Optionally, a detection apparatus can be configured to include a waste receptacle to which waste from the vessel is collected. For example, affinity reagents can be delivered from the apparatus through an ingress of a flow cell and waste can be removed through an egress of the flow cell to the apparatus. As such, a detection apparatus can be configured to deliver to a flow cell (or other vessel) affinity reagents that recognize one or more epitopes in a set of epitopes or set of standard polypeptides set forth herein.

One or more polypeptides (e.g. a standard polypeptide or test polypeptide) can be bound to affinity reagents. An affinity reagent can bind to an epitope in the amino acid sequence of a polypeptide. An affinity reagent that is bound to a polypeptide or otherwise used in a method set forth herein can have a label. A complex formed between a labeled affinity reagent and polypeptide can be detected by virtue of signals produced by the label. A complex between an affinity reagent and polypeptide can be in fluid-phase. Alternatively, a complex between an affinity reagent and polypeptide can be immobilized. For example, the polypeptide can be immobilized on a solid support via covalent bonding or another attachment mechanism set forth herein, and the affinity reagent can be immobilized via binding to the polypeptide. Thus, an affinity reagent can be attached to a solid support via binding to a polypeptide on the solid support. The opposite configuration can also occur, wherein an affinity reagent is immobilized on a solid support via covalent bonding or another attachment mechanism set forth herein, and a polypeptide is immobilized via binding to the affinity reagent. Thus, a polypeptide can be attached to a solid support via binding to an affinity reagent on the solid support. An immobilized complex can be detected via a label that is present on any member of the complex, such as a polypeptide or affinity reagent.

The present disclosure provides a plurality of polypeptides including one or more standard polypeptides having non-naturally occurring amino acid sequence(s) and one or more test polypeptides having naturally occurring amino acid sequence(s). In some configurations, the standard polypeptide(s) and test polypeptide(s) can be present in fluid-phase as a mixture. In other configurations, the standard polypeptide(s) and/or test polypeptide(s) can be immobilized. For example, the standard polypeptide(s) and test polypeptide(s) can be attached to addresses in an array. Optionally, a plurality of standard polypeptide(s) and test polypeptide(s) can be attached to structured nucleic acid particles such as those composed of nucleic acid origami.

In some cases, a plurality of polypeptides that includes one or more test polypeptides having naturally occurring amino acid sequences can include a plurality of different standard polypeptides, individual standard polypeptides of the set each including a non-naturally occurring amino acid sequence, wherein a set of different epitopes occurs in the set of different standard polypeptides, each of the different epitopes occurring in the non-naturally occurring amino acid sequence of a subset of the different standard polypeptides, and the non-naturally occurring amino acid sequence of each of the different standard polypeptides including a plurality of different epitopes of the set of epitopes. Optionally, a set of standard polypeptides can include two or more amino acid sequences set forth in SEQ ID Nos: 1 to 40.

Optionally, a plurality of polypeptides that includes one or more standard polypeptides can include a plurality of different test polypeptides from a proteome. The proteome can be obtained from any of a variety of organisms. Exemplary organisms from which a set of test polypeptides can be obtained include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. A polypeptide can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid.

Amino acid sequences present in one or more standard polypeptides can be non-native to one or more of the above organisms. For example, a plurality of polypeptides can include one or more test polypeptides having amino acid sequence(s) that are native to a particular organism (or other biological system) and can further include one or more standard polypeptides having amino acid sequence(s) that are not native to the particular organism (or other biological system). In some cases, a plurality of polypeptides that includes one or more test polypeptides having amino acid sequences that are native to a particular organism (or other biological system) can include a plurality of different standard polypeptides, individual standard polypeptides of the set each including an amino acid sequence that is non-native to the particular organism (or other biological system), wherein a set of different epitopes occurs in the set of different standard polypeptides, each of the different epitopes occurring in the non-native amino acid sequence of a subset of the different standard polypeptides, and the non-native amino acid sequence of each of the different standard polypeptides including a plurality of different epitopes of the set of epitopes. Optionally, the standard polypeptide(s) and test polypeptide(s) can be present in fluid-phase as a mixture, immobilized on solid phase, attached to (an) address(es) of an array, or attached to structured nucleic acid particle(s) such as nucleic acid origami.

A plurality of test polypeptides can include at least 1, 10, 100, 1×10⁶, 1×10⁹, 1 mole (6.02214076×10²³ molecules), or more polypeptide molecules. Alternatively or additionally, a plurality of polypeptides may contain at most 1 mole, 1×10⁹, 1×10⁶, 1×10⁴, 100, 10 or, 1 polypeptide molecule. A plurality of test polypeptides can include variety of different amino acid sequences. For example, the variety of full-length amino acid sequences in a plurality of test polypeptides can include substantially all different native-length amino acid sequences from a given organism or a subfraction thereof. A proteome or subfraction can have a complexity of at least 2, 5, 10, 100, 1×10³, 1×10⁴, 2×10⁴, 3×10⁴ or more different native-length amino acid sequences. Alternatively or additionally, a proteome or subfraction can have a complexity that is at most 3×10⁴, 2×10⁴, 1×10⁴, 1×10³, 100, 10, 5, 2 or fewer different native-length amino acid sequences.

The diversity of a proteome sample can include at least one representative for substantially all polypeptides encoded by the genome of the organism from which the sample was obtained, or a fraction thereof. For example, a plurality of test polypeptides may contain at least one representative for at least 60%, 75%, 90%, 95%, 99%, or more of the polypeptides encoded by a particular organism. Alternatively or additionally, a plurality of test polypeptides may contain a representative for at most 99%, 95%, 90%, 75%, 60% or less of the polypeptides encoded by a particular organism.

The present disclosure provides a method of preparing a polypeptide sample. The method can include steps of (a) obtaining a plurality of test polypeptides from an organism; and (b) contacting the plurality of test polypeptides with at least one standard polypeptide, thereby forming a polypeptide sample including the plurality of test polypeptides and the at least one standard polypeptide. Optionally, the at least one standard polypeptide includes an amino acid sequence selected from SEQ ID NOs: 1 to 40. Optionally, the at least one standard polypeptide includes a set of different standard polypeptides, individual standard polypeptides of the set each including a non-naturally occurring amino acid sequence, wherein a set of different epitopes occurs in the set of different polypeptides, each of the different epitopes occurring in the non-naturally occurring amino acid sequence of a subset of the different polypeptides, and the non-naturally occurring amino acid sequence of each of the different polypeptides including a plurality of different epitopes of the set of epitopes.

Test polypeptides can be obtained from an organism using methods known in the art. Test polypeptides can be extracted from cells, tissue, biological fluids or other sources using known techniques. Test polypeptides can optionally be separated or isolated from other components of the source. Standard polypeptides can be separated from biological components using the same methods. For example, one or more polypeptides can be separated or isolated from lipids, nucleic acids, hormones, enzyme cofactors, vitamins, metabolites, microtubules, organelles (e.g. nucleus, mitochondria, chloroplast, endoplasmic reticulum, vesicle, cytoskeleton, vacuole, lysosome, cell membrane, cytosol or Golgi apparatus), other polypeptides or the like. Polypeptide separation can be carried out using methods known in the art such as centrifugation (e.g. to separate membrane fractions from soluble fractions), density gradient centrifugation (e.g. to separate different types of organelles), precipitation, affinity capture, adsorption, liquid-liquid extraction, solid-phase extraction, chromatography (e.g. affinity chromatography, ion exchange chromatography, reverse phase chromatography, size exclusion chromatography, electrophoresis (e.g. polyacrylamide gel electrophoresis) or the like. Particularly useful polypeptide separation methods are set forth in Scopes, Polypeptide Purification Principles and Practice, Springer; 3rd edition (1993).

One or more standard polypeptides can be contacted with test polypeptides at any of a variety of stages in the extraction and separation of the test polypeptides. For example, a plurality of test polypeptides can be contacted with at least one standard polypeptide in fluid-phase, thereby forming a fluid-phase polypeptide sample including the plurality of test polypeptides and the at least one standard polypeptide. As such, one or more standard polypeptides can be co-fractionated with test polypeptides. As set forth herein, one or more standard polypeptides can be captured by solid support immobilization, for example, in the presence of test polypeptides. For example, a plurality of test polypeptides in fluid-phase can be contacted with at least one immobilized standard polypeptide, thereby forming an immobilized polypeptide sample including the plurality of test polypeptides and the at least one standard polypeptide. In another example, a plurality of immobilized test polypeptides can be contacted with at least one fluid-phase standard polypeptide, thereby forming an immobilized polypeptide sample including the plurality of test polypeptides and the at least one standard polypeptide. In certain configurations of the methods, an immobilized polypeptide sample is produced in the form of an array including addresses attached to standard polypeptides and addresses attached to test polypeptides.

The present disclosure provides a method of detecting polypeptides. The method can include steps of (a) obtaining a polypeptide sample including test polypeptides from an organism and at least one standard polypeptide; and (b) detecting at least one of the test polypeptides in the sample and detecting the at least one standard polypeptide in the sample. Optionally, the at least one standard polypeptide includes an amino acid sequence selected from SEQ ID NOs: 1 to 40. Optionally, the at least one standard polypeptide includes a set of two or more different standard polypeptides, individual standard polypeptides of the set each including a non-naturally occurring amino acid sequence, wherein a set of different epitopes occurs in the set of different polypeptides, each of the different epitopes occurring in the non-naturally occurring amino acid sequence of a subset of the different polypeptides, and the non-naturally occurring amino acid sequence of each of the different polypeptides including a plurality of different epitopes of the set of epitopes.

Polypeptides (e.g. a standard polypeptide or test polypeptide) can be detected using any of a variety of assays. For example, a polypeptide can be detected using one or more affinity reagents having binding affinity for the polypeptide. The affinity reagent and the polypeptide can bind each other to form a complex and, during or after formation, the complex can be detected. The complex can be detected directly, for example, due to a label that is present on the affinity reagent or polypeptide. In some configurations, the complex need not be directly detected, for example, in formats where the complex is formed and then the affinity reagent, polypeptide, or a label component that was present in the complex is subsequently detected.

Many polypeptide assays, such as enzyme linked immunosorbent assay (ELISA), achieve high-confidence characterization of one or more polypeptides in a sample by exploiting high specificity binding of affinity reagents to the polypeptide(s) and detecting the binding event while ignoring all other polypeptides in the sample. Binding assays can be carried out by detecting immobilized affinity reagents and/or polypeptides in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection assays employ SOMAmer reagents and SOMAscan assays commercialized by Soma Logic (Boulder, CO). In one configuration, a sample is contacted with aptamers that are capable of binding polypeptides with specificity for the amino acid sequence of the polypeptides. The resulting aptamer-polypeptide complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

Exemplary assay formats that can be performed at a variety of plexity scales up to and including proteome scale are set forth in U.S. Pat. No. 10,473,654; US Pat. App. Pub. Nos. 2020/0318101 A1, 2020/0286584 A1 or 2023/0114905 A1; or Egertson et al., BioRxiv (2021), DOI: 10.1101/2021.10.11.463967, each of which is incorporated herein by reference. A plurality of polypeptides can be assayed for binding to affinity reagents, for example, on single-molecule resolved polypeptide arrays. Standard polypeptides can be included in the assay, for example, being attached to addresses in an array of test polypeptides. Polypeptides (e.g. a standard polypeptide or test polypeptide) can be in a denatured state or native state when manipulated or detected in a method set forth herein.

Turning to the example of an array-based configuration, the identity of the test polypeptide at any given address is typically not known prior to performing the assay. The location and identity of one or more standard polypeptides may be known or unknown prior to performing the assay. The assay can be used to identify polypeptides (e.g. a standard polypeptide or test polypeptide) at one or more addresses in the array. A plurality of affinity reagents, optionally labeled (e.g. with fluorophores), can be contacted with the array, and the presence of affinity reagents can be detected from individual addresses to determine binding outcomes. A plurality of different affinity reagents can be delivered to the array and detected serially, such that each cycle detects binding outcomes for an individual affinity reagent. In some configurations, a plurality of affinity reagents can be detected in parallel, for example, when different affinity reagents are distinguishably labeled.

In particular configurations, the methods can be used to identify a number of different polypeptides that exceeds the number of affinity reagents used. For example, the number of polypeptides identified can be at least 5×, 10×, 25×, 50×, 100× or more than the number of affinity reagents used. This can be achieved, for example, by (1) using promiscuous affinity reagents that bind to multiple different polypeptides suspected of being present in a given sample, and (2) subjecting the polypeptide sample to a set of promiscuous affinity reagents that, taken as a whole, are expected to bind each polypeptide in a different combination, such that each polypeptide is expected to generate a unique profile of binding and non-binding events. Promiscuity of an affinity reagent can arise due to the affinity reagent recognizing an epitope that is known to be present in a plurality of different polypeptides. For example, epitopes having relatively short amino acid lengths such as dimers, trimers, tetramers or pentamers can be expected to occur in a substantial number of different polypeptides in a typical proteome. Alternatively or additionally, a promiscuous affinity reagent may recognize different epitopes (e.g. epitopes differing from each other with regard to amino acid composition or sequence). For example, a promiscuous affinity reagent that is designed or selected for its affinity toward a first trimer epitope may bind to a second epitope that has a different sequence of amino acids compared to the first epitope.

Although performing a single binding reaction between a promiscuous affinity reagent and a complex polypeptide sample may yield ambiguous results regarding the identity of the different polypeptides to which it binds, the ambiguity can be resolved by decoding the binding profiles for each polypeptide using machine learning or artificial intelligence algorithms that are based on probabilities for the affinity reagents binding to candidate polypeptides. For example, a plurality of different promiscuous affinity reagents can be contacted with a complex population of polypeptides, wherein the plurality is configured to produce a different binding profile for each candidate polypeptide suspected of being present in the population. The plurality of promiscuous affinity reagents can produce a binding profile for each individual polypeptide that can be decoded to identify a unique combination of positive (i.e. observed binding events) and/or negative binding outcomes (i.e. observed non-binding events), and this can in turn be used to identify the individual polypeptide as a particular candidate polypeptide having a high likelihood of exhibiting a similar binding profile.

Binding profiles can be obtained for test polypeptides and/or standard polypeptides and decoded. In many cases one or more binding events produces inconclusive or even aberrant results and this, in turn, can yield ambiguous binding profiles. For example, observation of binding outcome at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate polypeptides that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate polypeptides. A binding model can include information regarding the probability or likelihood of a given candidate polypeptide generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate polypeptides using a binding model. For example, to identify an unknown polypeptide in a sample, an empirical binding profile for the polypeptide can be compared to results computed by the binding model for many or all candidate polypeptides suspected of being in the sample. A machine learning or artificial intelligence algorithm can be used. An algorithm used for decoding can utilize Bayesian inference. In some configurations, identity for an unknown polypeptide is determined based on a likelihood of the unknown polypeptide being a particular candidate polypeptide given the empirical binding pattern or based on the probability of a particular candidate polypeptide generating the empirical binding pattern. Particularly useful decoding methods are set forth, for example, in U.S. Pat. No. 10,473,654; US Pat. App. Pub. Nos. 2020/0318101 A1 or 2023/0114905 A1, or Egertson et al., BioRxiv (2021), DOI: 10.1101/2021.10.11.463967, each of which is incorporated herein by reference. A method of the present disclosure can be configured to identify at least one test polypeptide from an organism based on known identity of at least one standard polypeptide. For example, results of decoding a test polypeptide can be compared to results of decoding a standard polypeptide.

One or more compositions set forth herein can be provided in kit form including, if desired, a suitable packaging material. In one configuration, for example, a particle, solid support, flow cell, array, standard polypeptide, affinity reagent, assay reagent and/or other composition set forth herein can be provided in one or more vessels. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular polypeptide or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

### EXAMPLE I

### Design of Standard Polypeptide Sequences Having Epitopes for Affinity Reagents

This example demonstrates generation of synthetic polypeptide sequences for use as standard polypeptides. The standard polypeptides can be used as target reagents (e.g. bait) for identifying and/or validating affinity reagents, for example, in a binding assay or screen. The standard polypeptides can also be used as controls in a binding assay, wherein binding of affinity reagents to unknown polypeptides is evaluated relative to binding of the affinity reagents to the standard polypeptides.

Polypeptide sequences were generated by an algorithm that utilized a graph structure and having the following two main parts:

(1) Generating an epitope graph; and

(2) Traversing the epitope graph. In the first part, a directed graph was generated from a list of epitopes using the Networkx Python package (available on the worldwide web at network.org) where the nodes represented epitopes and the edges between nodes represented an adjacency between nodes given some allowed overlap. Directionality of the epitopes was set such that the epitope “NAV” was different than its reverse “VAN”. In the second part, the nodes in the graph were stochastically stepped through. Each step included: 1) incorporating the current node into the sequence; 2) selecting a valid edge to traverse; 3) traversing the edge and selecting the next node; and 4) removing the current node and its edges from the graph. A more detailed explanation follows.

Generating an epitope graph

The main function generate graph took the following arguments:
epitope_len (int): the length of each epitope in the sequences (e.g. 3->trimers),
epitope_rep (int): the minimum number of times each epitope should be represented in unique sequences,
overlap (int): how many amino acids are adjacent epitopes allowed to overlap,
epitope_list (list): OPTIONAL. If not all possible epitopes are desired a graph will be generated only from the epitopes passed in, and
FASTA (pathlib.Path) OPTIONAL. Path to a FASTA file from which edge weights are determined based on the transition frequency between adjacent epitopes (given an overlap); and returned
graph: a directed graph (networkx.DiGraph).

A suffix was appended to each epitope to facilitate representation of each epitope multiple times. For example, the epitope AGM was represented three times as AGM_000, AGM_001, and AGM_002. This is done since each node needs to have a unique identity.

For each epitope the algorithm determined each possible adjacent epitope given overlap. For epitope_len=3; overlap=2, the possible adjacent epitopes for AGM would be GMA, GMC, GMD, GME, . . . , GMY (i.e. 20 different trimer sequences in which the third amino acid is any one of the 20 standard amino acids).

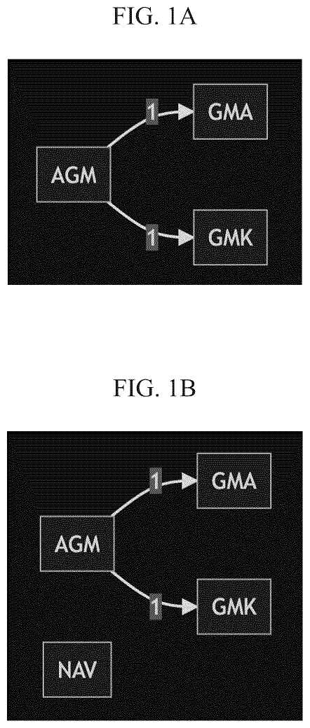

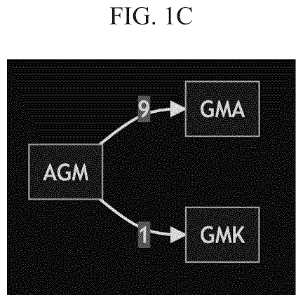

Then AGM was paired with each of the possible adjacent epitopes, and a check was made to determine if both exist in the epitope list. If so, a node was created for AGM and each of its adjacent epitopes and a directed edge was added in the graph from the AGM node to each adjacent epitope node. For instance, the graph shown in FIG. 1A results if the epitope list is [“AGM”, “GMA”, “GMK”] and epitope_len=3; overlap=2.

Given a limited epitope set, it may not be possible to connect all epitopes/nodes by edges. These nodes are added as singletons to the graph. For example, for the epitope list [“GM”, “GMA”, “GMK”, “NAV”] and epitope_len=3; overlap=2 the graph appeared as shown in FIG. 1B. Then, sequences from an input FASTA file were parsed into their constituent epitopes and the number of transitions between epitopes were counted and added to the appropriate edge weights in the graph. So, for the epitope list [“AGM”, “GMA”, “GMK”]. epitope_len=3 and overlap=2 an observation of AGM ->GMA represented by the amino acid sequence AGMA occurred 8 times and an observation of AMG ->GMK or AGMK never occurred, giving the graph shown in FIG. 1C.

The initial graph generation of connecting all possible node transitions given an input epitope_len and overlap by directed edges was observed to serve as a pseudocount if the transition did not occur in the FASTA. This ensures that all transitions have a non-zero probability of occurring even if they do not occur in the input FASTA. The final graph was saved as a .gml file.

Traversing the graph

The main function traverse graph took the following arguments:
graph: the graph that was generated in the previous part,
path len: the maximum length of a sequence/peptide (length of path through the graph), and
n_paired: the maximum amount of times any two epitopes can co-occur within a sequence; and returned
paths: a list of strings representing the generated synthetic peptides.

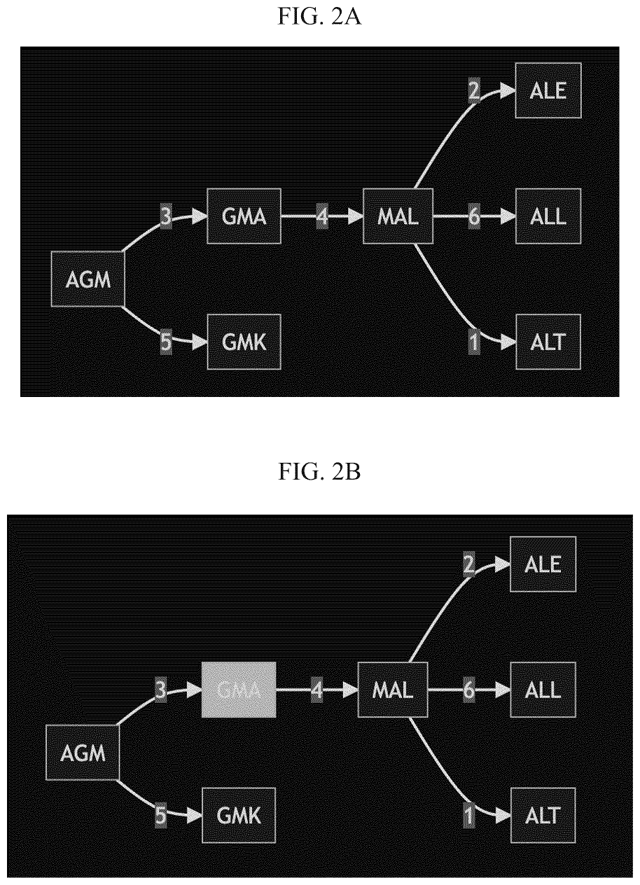

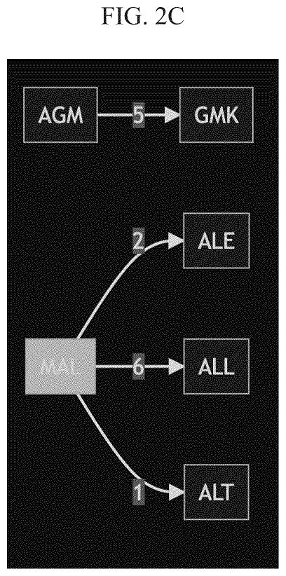

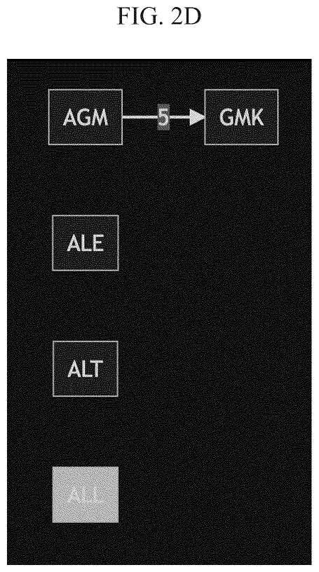

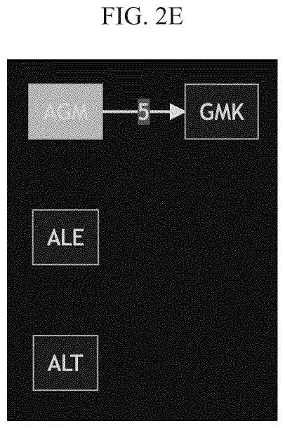

A graph through which a basic traversal will be detailed is illustrated in FIG. 2A. The graph was generated with epitope_len=3 and overlap=2. The process starts with an empty sequence A random node in the graph was selected and added to a sequence. For example, the node GMA was selected as shown in FIG. 2B.

GMA was added to the sequence to get “GMA.” From the GMA node the edge weights for all outgoing edges were taken and generated a probability of traversing any of those edges was generated. An edge to traverse was then selected based on those probabilities. For this node there was only one edge to traverse, so it was taken with probability 1, node MAL was selected and the previous node was deleted as shown in FIG. 2C.

MAL was added to the sequence with the appropriate overlap to get “GMAL.” From the MAL node multiple edges were now available to traverse. The probability of going to node ALE was 2/(2+6+1)=0.22, node ALL 6/(2+6+1)=0.66, and node ALT 1/(2+6+1) 0.11. The edge to node ALL can be stochastically chosen, as shown in FIG. 2D.

ALL was added to the sequence with the appropriate overlap to get “GMALL.” From the ALL node there were no outgoing edges. Because of this the requirement for an overlap was removed, so the sequence could continue to extend. This is called a “jump” and constitutes selecting a random node in the graph. Node AGM was selected as shown in FIG. 2E, and the process continued.

AGM was added to the sequence without an overlap due to the jump to get “GMALLAGM.” From the AGM node, the process continued for a given sequence until adding a node by edge traversal or by jumping made the sequence longer than path len. That sequence was then appended to the output paths list and the traversal started over with a new sequence.

When all nodes were removed from the graph, the algorithm immediately exited after padding the final path with random sequence. In addition to this traversal, two main restrictions were applied when selecting an edge to traverse or when selecting a random node in a jump. First an epitope was not allowed to appear in a sequence more than once. Second, any pair of epitopes was not allowed to co-occur in a sequence any more than n paired times. If no nodes remained in the graph that would meet these two conditions, a random epitope that would meet the conditions was generated.

Target Epitopes

Table I shows the epitope targets that were used to generate standard polypeptides. Amino acids are indicated by the single letter code. Gaps in epitope sequences are indicated by the symbol X, which can be any amino acid residue. When generating the standard polypeptides trimer epitopes were treated as is. Tetramers were split into their component trimers. Each of these groups (represented as trimers) were deduplicated and represented three times each in the library.

TABLE I

| | | | | | | |
|---|---|---|---|---|---|---|
| HHH | FHH | FRW | ATD | TXQV | RDE | PHS |
| RYYR | RWY | RFF | EDXE | TQQ | DTV | YTD |
| HYH | WFR | RHHR | EXLT | EXKE | ETR | DRP |
| HRY | HRH | KFR | TTXV | SEXS | PAY | ETY |
| RFFR | YRY | RFK | DAR | EXYD | WSP | HPD |
| HHR | YHR | YFR | LSXE | VHT | DDS | TVP |
| FHR | WRW | RWWR | DED | LXEN | FST | YPS |
| RHRH | RRYF | WLR | DMXA | RGR | PSW | |
| RWR | RFRF | TTR | TXRD | WNK | QSE | |
| RFW | HWW | AIT | QXLE | LEEL | SPY | |
| YRHR | FRYF | DTT | DNT | EDRY | DDY | |
| YYR | FRRF | AQD | DSD | YWL | DPY | |
| WFH | FRR | EMQ | MET | DTR | HSP | |

Standard Polypeptide Sequences

Given a peptide path_len of 50, an epitope_len of 3, an overlap of 2, epitope_rep of 3 and with no restriction on n_paired and using a FASTA file containing human cytosolic polypeptides, the 14 standard polypeptides shown in Table II were generated. A standard polypeptide was passed if it had a solubility within a predefined range, such as a normalized score of over 0.5 using protein-sol (protein- sol.manchester.ac.uk).

TABLE II

| | | | | SEQ ID |
|---|---|---|---|---|
| Name | Sequence | Solubility | pI | NO |
| E1-01 | FTVPPHSPYVHTTTRFRFWDRPDMDAR | 0.575 | 11.560 | 1 |
| | WFRYYRHHRYFRWYRFKRFFRRY | | | |
| E1-02 | PSWENPHSPYKFRRFFRYFRWRWYYPS | 0.600 | 10.520 | 2 |
| | DEDAEFHHRHHHRRYYRHRYVRD | | | |
| E1-03 | QYLTGRDEDEELSNEDKERHHRYYRYF | 0.565 | 6.930 | 3 |
| | RWWRWRDYWLRFHHHDNTVPTQQ | | | |
| E1-04 | WNKKFRYFRRFRFWDDSDFHHHTGRT | 0.575 | 11.650 | 4 |
| | WTVPFHRHRETYRLTATDRGRWFH | | | |
| E1-05 | QSEPSWMFAAQVSELFFRFRATDYRHR | 0.569 | 9.640 | 5 |
| | HHPDLVENKFRHSPYDRPDRY | | | |
| E1-06 | AQDETYYRYDRYDDSDTNTRDEDRPLS | 0.670 | 4.800 | 6 |
| | WPHSDPYYWLRHYHRLMETLQV | | | |
| E1-07 | ETYEMQDTTRQKLEELRRFFDPYRFRSE | 0.723 | 4.940 | 7 |
| | VSYHRDMFAQDDTRSFEDAR | | | |
| E1-08 | DARDRYEWKELWEESYDTAQDHWWR | 0.670 | 4.990 | 8 |
| | WYEMQRWWVHTEKKEMETRDTVR | | | |
| E1-09 | PAYRFKYTDAITDDYWNKPSWLEELYW | 0.602 | 7.840 | 9 |
| | LRDTTRKQVRGRHPDWSPEDR | | | |
| E1-10 | DDYEDRHPDWFRYLEEWSPDPYQSEER | 0.723 | 4.330 | 10 |
| | KEEMQYRYDTTMTALSFDNTRWR | | | |
| E1-11 | DTVWNKWFHRYHRWFRWWRAITYPSD | 0.550 | 8.130 | 11 |
| | TRHWWQSEEYLFSTDSDTKQDNT | | | |
| E1-12 | TLTEDKHWWATDWSPERLFSTYTDHYH | 0.602 | 6.050 | 12 |
| | RWWTWRPAYTFTYPSTQQDMT | | | |
| E1-13 | RGRRDEVHTHYHTQQYTDRFWFHRETR | 0.569 | 6.820 | 13 |
| | FYDPAYFSTAITFLEDTRMET | | | |
| E1-14 | DDYDDSRRYWFHMENEFYDTVETRQFL | 0.722 | 5.230 | 14 |
| | RFKQFVTTRKYENLEDTKKMI | | | |

Table III lists all target epitopes that occur in at least three different standard polypeptides. The epitopes are listed in the first column (“Epitope”) and the second through fourteenth columns identify presence (indicated by “T”) or absence (indicated by “F”) for the epitope targets in the respective standard polypeptides. The standard polypeptides are identified by E1-01 through E1-14 labels as used in Table II. The final column (“SP_num”) provides a count of the number of standard polypeptides that include each target epitope. The final row (“Ep_num”) provides a count of the number of target epitopes in each standard polypeptide. As shown, the number of target epitopes per standard polypeptide ranged from 7 to 21.

TABLE III

| | E1- | E1- | E1- | E1- | E1- | E1- | E1- | E1- | E1- | E1- | E1- | E1- | E1- | E1- | SP_ |
|---|---|---|---|---|---|---|---|---|---|---|---|---|---|---|---|
| Epitope | 01 | 02 | 03 | 04 | 05 | 06 | 07 | 08 | 09 | 10 | 11 | 12 | 13 | 14 | num |
| FRW | 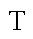 | 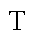 | 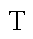 | F | F | F | F | F | F | F | 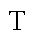 | F | F | F | 4 |
| YYR | 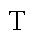 | 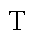 | 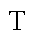 | F | F | 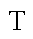 | F | F | F | F | F | F | F | F | 4 |
| YFR | 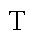 | 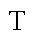 | 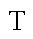 | 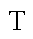 | F | F | F | F | F | F | F | F | F | F | 4 |
| HRY | 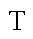 | 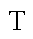 | 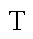 | F | F | F | F | F | F | F | 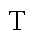 | F | F | F | 4 |
| YHR | F | F | F | F | F | 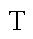 | 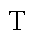 | F | F | F | 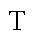 | 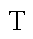 | F | F | 4 |
| TTR | 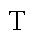 | F | F | F | F | F | 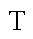 | F | 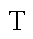 | F | F | F | F | 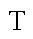 | 4 |
| WFH | F | F | F | 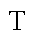 | F | F | F | F | F | F | 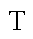 | F | 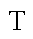 | 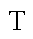 | 4 |
| DED | F | 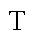 | 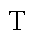 | F | F | 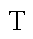 | F | F | F | F | F | F | F | F | 3 |
| WRW | F | 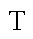 | 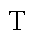 | F | F | F | F | 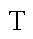 | F | F | F | F | F | F | 3 |
| RYYR | 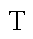 | 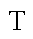 | 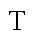 | F | F | F | F | F | F | F | F | F | F | F | 3 |
| KFR | F | 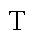 | F | 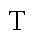 | 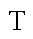 | F | F | F | F | F | F | F | F | F | 3 |
| RFK | 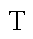 | F | F | F | F | F | F | F | 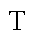 | F | F | F | F | 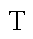 | 3 |
| FRR | 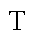 | 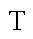 | F | 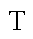 | F | F | F | F | F | F | F | F | F | F | 3 |
| RFW | 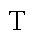 | F | F | 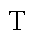 | F | F | F | F | F | F | F | F | 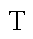 | F | 3 |
| WFR | 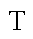 | F | F | F | F | F | F | F | F | 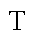 | 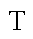 | F | F | F | 3 |
| HHH | F | 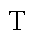 | 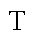 | 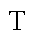 | F | F | F | F | F | F | F | F | F | F | 3 |
| HYH | F | F | F | F | F | 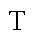 | F | F | F | F | F | 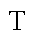 | 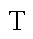 | F | 3 |
| HHR | 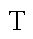 | 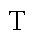 | 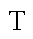 | F | F | F | F | F | F | F | F | F | F | F | 3 |
| RWR | F | 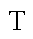 | 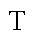 | F | F | F | F | F | F | 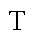 | F | F | F | F | 3 |
| HRH | F | 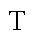 | F | 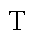 | 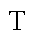 | F | F | F | F | F | F | F | F | F | 3 |
| FHR | F | F | F | 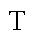 | F | F | F | F | F | F | 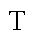 | F | 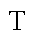 | F | 3 |
| YRY | F | F | 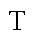 | F | F | 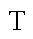 | F | F | F | 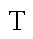 | F | F | F | F | 3 |
| WLR | F | F | 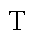 | F | F | 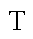 | F | F | 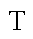 | F | F | F | F | F | 3 |
| WNK | F | F | F | 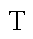 | F | F | F | F | 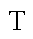 | F | 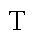 | F | F | F | 3 |
| YWL | F | F | 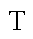 | F | F | 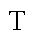 | F | F | 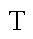 | F | F | F | F | F | 3 |
| DTR | F | F | F | F | F | F | 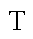 | F | F | F | 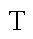 | F | 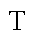 | F | 3 |
| FST | F | F | F | F | F | F | F | F | F | F | 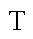 | 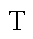 | 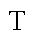 | F | 3 |
| RDE | F | F | 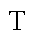 | F | F | 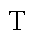 | F | F | F | F | F | F | 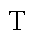 | F | 3 |
| DTV | F | F | F | F | F | F | F | 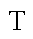 | F | F | 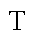 | F | F | 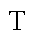 | 3 |
| WSP | F | F | F | F | F | F | F | F | 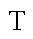 | 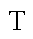 | F | 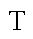 | F | F | 3 |
| DDS | F | F | F | 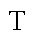 | F | 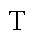 | F | F | F | F | F | F | F | 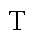 | 3 |
| PSW | F | 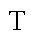 | F | F | 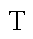 | F | F | F | 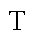 | F | F | F | F | F | 3 |
| QSE | F | F | F | F | 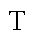 | F | F | F | F | 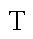 | 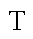 | F | F | F | 3 |
| SPY | 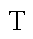 | 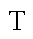 | F | F | 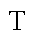 | F | F | F | F | F | F | F | F | F | 3 |
| DDY | F | F | F | F | F | F | F | F | 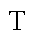 | 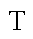 | F | F | F | 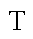 | 3 |
| DPY | F | F | F | F | F | 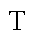 | 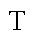 | F | F | 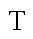 | F | F | F | F | 3 |
| HSP | 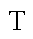 | 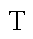 | F | F | 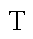 | F | F | F | F | F | F | F | F | F | 3 |
| HPD | F | F | F | F | 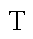 | F | F | F | 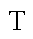 | 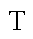 | F | F | F | F | 3 |
| TVP | 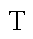 | F | 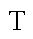 | 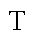 | F | F | F | F | F | F | F | F | F | F | 3 |
| YPS | F | 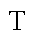 | F | F | F | F | F | F | F | F | 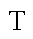 | 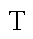 | F | F | 3 |
| DTT | F | F | F | F | F | F | 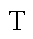 | F | 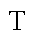 | 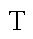 | F | F | F | F | 3 |
| DSD | F | F | F | 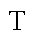 | F | 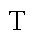 | F | F | F | F | 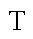 | F | F | F | 3 |
| ATD | F | F | F | 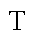 | 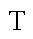 | F | F | F | F | F | F | 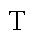 | F | F | 3 |
| DNT | F | F | 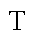 | F | F | F | F | F | F | 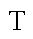 | 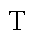 | F | F | F | 3 |
| DAR | 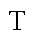 | F | F | F | F | F | 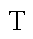 | 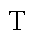 | F | F | F | F | F | F | 3 |
| EMQ | F | F | F | F | F | F | 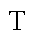 | 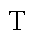 | F | 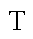 | F | F | F | F | 3 |
| TQQ | F | F | 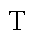 | F | F | F | F | F | F | F | F | 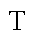 | 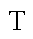 | F | 3 |
| AQD | F | F | F | F | F | 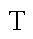 | 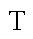 | 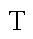 | F | F | F | F | F | F | 3 |
| FHH | F | 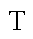 | 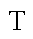 | 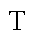 | F | F | F | F | F | F | F | F | F | F | 3 |
| RWY | 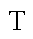 | 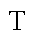 | F | F | F | F | F | 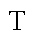 | F | F | F | F | F | F | 3 |
| HWW | F | F | F | F | F | F | F | 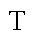 | F | F | 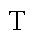 | 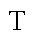 | F | F | 3 |
| RFF | 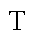 | 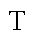 | F | F | F | F | 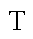 | F | F | F | F | F | F | F | 3 |
| AIT | F | F | F | F | F | F | F | F | 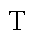 | F | 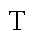 | F | 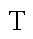 | F | 3 |
| VHT | 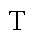 | F | F | F | F | F | F | 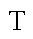 | F | F | F | F | 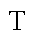 | F | 3 |
| RGR | F | F | F | 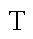 | F | F | F | F | 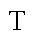 | F | F | F | 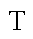 | F | 3 |
| ETR | F | F | F | F | F | F | F | 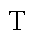 | F | F | F | F | 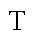 | 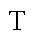 | 3 |
| PAY | F | F | F | F | F | F | F | F | 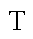 | F | F | 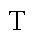 | 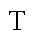 | F | 3 |
| PHS | 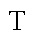 | 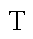 | F | F | F | 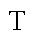 | F | F | F | F | F | F | F | F | 3 |
| YTD | F | F | F | F | F | F | F | F | 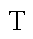 | F | F | 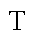 | 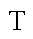 | F | 3 |
| DRP | 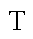 | F | F | F | 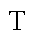 | 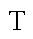 | F | F | F | F | F | F | F | F | 3 |
| ETY | F | F | F | 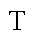 | 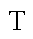 | 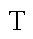 | F | F | F | F | F | F | F | F | 3 |
| E_num | 20 | 21 | 18 | 16 | 10 | 15 | 9 | 9 | 14 | 11 | 16 | 10 | 14 | 7 | |

### EXAMPLE II

### A Second Set of Standard Polypeptide Sequences

A second set of standard polypeptides was generated for a second set of epitopes using methods set forth in Example I.

Further criteria for passing amino acid sequences for the second set included: if normalized solubilities for any standard polypeptides was below 0.500 it was rejected and if was above 0.600 it was accepted. If normalized solubilities for the standard polypeptides were between 0.500-0.600 then the pI for the standard polypeptides had to be in the range of 4.0 to 6.6 or 7.4 to 10.0. The pI ranges were chosen to yield amino acid sequences with pI that differed from 7.0 by at least 0.4 and by at most 2.6.

The resulting set of standard polypeptides is shown in Table IV.

TABLE IV

| | | | | SEQ ID |
|---|---|---|---|---|
| Name | Sequence | Solubility | pI | NO |
| E2-00 | AHAWYYFYHWYHHLYRHFYDSRHWH | 0.643 | 7.00 | 15 |
| | FHYFDSTHTSHRIHSDEDDERHPRF | | | |
| E2-01 | RFVRPRHLFPRFQHVRDSDEDDEAQDA | 0.727 | 11.10 | 16 |
| | TTSRHIRYWQVRNRYMQRPAV | | | |
| E2-02 | DTSFRYWHYFATITTEYHWHFYHHHRI | 0.555 | 9.76 | 17 |
| | HFRPFRNYERDDARVFMRWYH | | | |
| E2-03 | RPHRPWLHVRIRYWRMHFVRMQRFYH | 0.631 | 11.91 | 18 |
| | WYHHSSHDARVPAVFHYFWHHQVR | | | |
| E2-04 | QPTPRHPRYRTTSFMRHTSDDDEDDTPT | 0.643 | 6.49 | 19 |
| | PHRVHRPHTYSPAVDSDEFRPF | | | |
| E2-05 | HMRRNFISQAYAPRYDDTPTSSTHTSWH | 0.587 | 9.88 | 20 |
| | HRLYRDYEDHVRASWDSRFRY | | | |
| E2-06 | AYADDDTSDAARTTDHIRYSYSTHTHN | 0.505 | 9.74 | 21 |
| | SARVPFRPHRPFDSRHRIHTQQ | | | |
| E2-07 | SYSFRNYQPTFVRAYDTTSRWFDYERM | 0.555 | 9.76 | 22 |
| | HWHFYAMRMTATYSEMQRLHV | | | |
| E2-08 | EMQRHLRNFERDATIISQAMRDSTHTTE | 0.643 | 10.67 | 23 |
| | PRHPRFQHRVFRNYDNTTDPRY | | | |
| E2-09 | AHAATTDAQDDHVDTTEFHYRNFDDD | 0.670 | 4.71 | 24 |
| | ATDWYYSSTEMQHRVPFRYRWF | | | |
| E2-10 | RHLQVRFPRFHYQPTDSDATTNRYRMH | 0.551 | 9.89 | 25 |
| | HNSSYSFMRTQQDSTHWYYDTT | | | |
| E2-11 | RFQRPWDNTTSSTMTARTTRLYRDQDD | 0.627 | 11.82 | 26 |
| | AAASWFPRNRYTPTAMRRFVRHI | | | |
| E2-12 | ERDHIRHMRTQQASWATIHNSAQDDHV | 0.771 | 5.93 | 27 |
| | AYDDTTLHVRHIDTSSSHDQD | | | |
| E2-13 | TYSMTASSHDAARWFNTTATDRPWDN | 0.602 | 6.63 | 28 |
| | TWHHDYEHWHAYARLYDTPHMR | | | |
| E2-14 | ISQDARDQDRFVATDAYDAHAMNMKPI | 0.747 | 4.54 | 29 |
| | IMAQWRPQQPDPMELRQNYID | | | |

### EXAMPLE III

### Binding of Standard Polypeptide to a Panel of Affinity Reagents

This example demonstrates binding of a standard polypeptide to a plurality of affinity reagents and use of a decoding algorithm to confirm the identity of the standard polypeptide

Epi 4 polypeptides having the sequence of E1-04 (SEQ ID NO: 4) were obtained from a commercial source. The polypeptides were modified with biotin and bound to nucleic acid origami tiles, each tile having a single streptavidin moiety, to form SNAP-Ps (polypeptide-attached structured nucleic acid particles). The SNAP-Ps were attached to a solid support to form an array of individually resolvable polypeptides. SNAP-Ps were made and attached to the array using methods set forth in US Pat. App. Pub. No. 2022/0290130 A1, which is incorporated herein by reference. The array also included control SNAPs (“Strep Tile”) having streptavidin with no polypeptide attached.

A set of 30 different Lobes (labeled probes) was prepared as follows. For each Lobe type, multiple copies of the same affinity reagent were attached to an origami nucleic acid tile. As such, each Lobe had primary affinity for the same epitope and also had increased binding avidity due to the presence of multiple affinity reagents. Each Lobe also contained multiple fluorescent labels to allow for increased signal. Lobes were formed as set forth in US Pat. App. Pub. No. 2022/0162684 A1, which is incorporated herein by reference. Table V shows the primary epitope targets for each of the Lobes and the type of affinity reagent attached to each Lobe (i.e. aptamers or full-length antibodies). The Lobe marked as ‘control’ included an origami tile with no attached affinity reagent.

TABLE V

| Primary Epitope Target | Affinity Reagent Type |
|---|---|
| WFR | Aptamer |
| HSP | Antibody |
| HPD | Antibody |
| YPS | Antibody |
| HHH | Aptamer |
| DTV | Antibody |
| DTR | Antibody |
| RWWR | Aptamer |
| HRH | Aptamer |
| YWL | Antibody |
| RHRH | Aptamer |
| FST | Antibody |
| YFR | Aptamer |
| RDE | Antibody |
| WNK | Antibody |
| HFR | Aptamer |
| FRRF | Aptamer |
| PSW | Antibody |
| RFFR | Aptamer |
| RFRF | Aptamer |
| WSP | Antibody |
| WYY | Aptamer |
| RYY | Aptamer |
| YRY | Aptamer |
| Control | none |

The array of SNAP-Ps was serially contacted with the Lobes listed in Table V as set forth in Egertson et al., BioRxiv (2021), DOI: 10.1101/2021.10.11.463967 and U.S. patent application Ser. No. 18/045,036, each of which is incorporated herein by references, with the following conditions. For each cycle the following steps were performed (1) a single Lobe type was introduced to the array and allowed to incubate at room temperature for 30 minutes, (2) non-bound Lobes were removed by washing, (3) the array was detected using a fluorescence detector, (4) Lobes were removed from the array by treatment with 6M Guanidinium Chloride, (5) the array was detected a second time, and the next cycle was then performed.

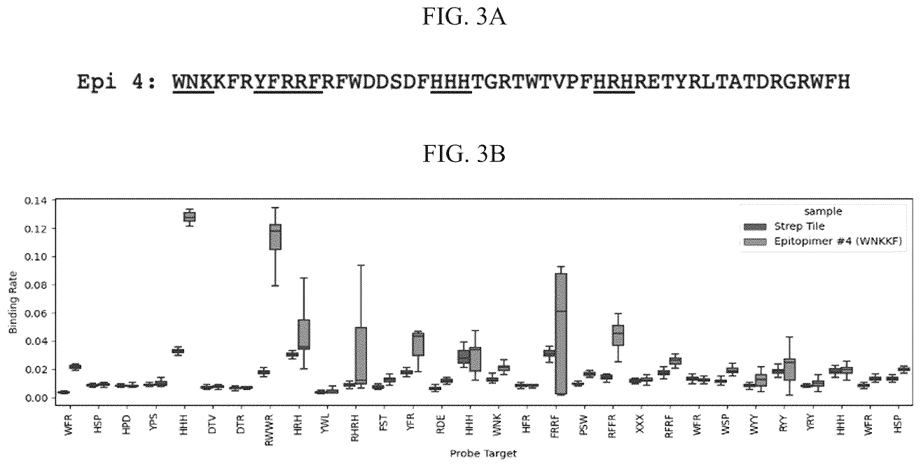

Binding rates were determined for the binding of each Lobe to each address on the array as set forth in Egertson et al., BioRxiv (2021), DOI: 10.1101/2021.10.11.463967 and U.S. patent application Ser. No. 18/045,036, each of which is incorporated herein by reference. FIG. 3A shows the sequence for the Epi 4 standard polypeptide (SEQ ID NO:4) and the locations of 5 epitopes targeted by the Lobes are indicated by underlines. FIG. 3B shows a box plot of binding rates observed for each of the 30 cycles. The cycles are identified on the x-axis according to the target epitope for the Lobe delivered in the cycle. A pair of binding rates is presented for each Lobe including binding to the Strep Tile (left side, dark shaded boxes) and binding to the Epi 4 standard polypeptide (right side, light shaded boxes).

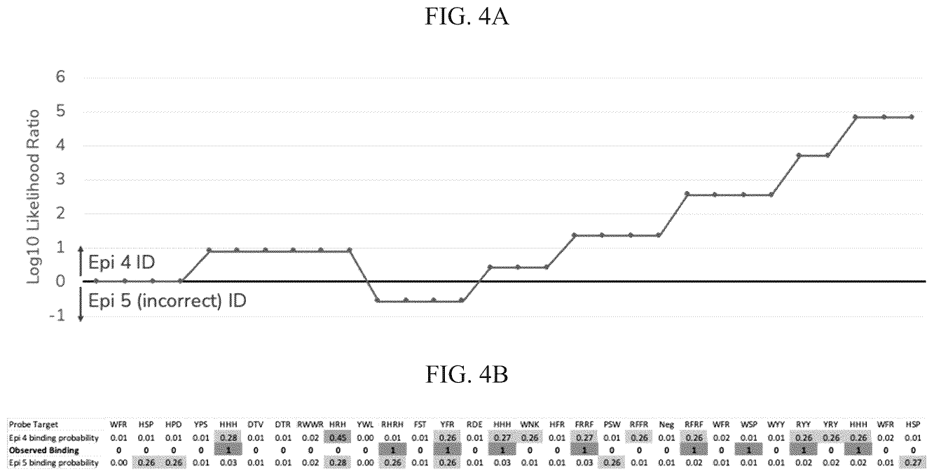

The binding rates were evaluated using the decoding algorithm set forth in Egertson et al., BioRxiv (2021), DOI: 10.1101/2021.10.11.463967 and U.S. patent application Ser. No. 18/045,036, each of which is incorporated herein by reference. The decoding algorithm was configured to determine the likelihood of each address in the array containing the Epi 4 polypeptide or the Epi 5 standard polypeptide (i.e. E1-05 (SEQ ID NO: 5). The Epi 5 standard polypeptide was selected as a decoding control because (a) it was not present in the array and (b) it has a relatively close a priori binding profile compared to the a priori binding profile for Epi 4. Decoding was performed serially using the results of the 30 cycles. FIG. 4A plots the log10 likelihood ratio for the polypeptides on the array being identified (“ID”) as Epi 4 (correct) vs. Epi 5 (incorrect) for each decoded cycle. The points on the plotted line correlate with the respective cycles shown in FIG. 4B. Also shown in FIG. 4B are the a priori binding probabilities for each Lobe binding to either the Epi 4 or Epi 5 polypeptides and the observed binding in binary values with “1” indicating binding observed above a threshold value, and “0” indicating lack of binding above the threshold value.

The results for the first three cycles (WFR, HSP and HPD) indicated that non-binding outcomes had negligible impact on the likelihoods. The results of the RHRH cycle indicated that spurious binding outcomes matching Epi 5 increased the likelihood of an incorrect identification, but did not prevent an eventual correct identification. The results of the YFR cycle indicated that binding outcomes matching both standard polypeptides had negligible impact on the accuracy of the likelihoods. The results of the WSP cycle indicated that binding outcomes matching neither standard polypeptide also had negligible impact on the accuracy of the likelihoods. Ultimately, the results obtained from all cycles indicated that Epi 4 was 100,000 times more likely to be the correct identification compared to an Epi 5 (incorrect) identification.

### EXAMPLE IV

### A Third Set of Standard Polypeptide Sequences

A third set of standard polypeptides was generated and included the sequences listed in Table VI.

TABLE VI

| | | | | SEQ ID |
|---|---|---|---|---|
| Name | Sequence | Solubility | pI | NO |
| EPT15 | FREWNKFRRFSLFFSTFWRLEFPFSHPDF | 0.627 | 12.11 | 30 |
| | RFLPQTIPRFRFWLRDRIWSP | | | |
| EPT16 | HFHFRNWEVHHHDTRHRHWFRDPYPS | 0.663 | 6.88 | 31 |
| | WQLPFISRDEDTVHSPSVHETY | | | |
| KJE01 | GGAAGGHPDGGAAGGFHSGGAAGGDP | 0.675 | 6.20 | 32 |
| | YGGAAGGDTRGGAAGGHHHHGG | | | |
| KJE02 | GGAAGGHSPGGAAGGDFPGGAAGGYF | 0.742 | 6.62 | 33 |
| | SGGAAGGETHGGAAGGHHHHGG | | | |
| KJE03 | GGAAGGHPDGGFHSGGDPYGGDTRGG | 0.676 | 6.62 | 34 |
| | HHHHGG | | | |
| KJE04 | GGAAGGHSPGGDFPGGVFSGGETHGGH | 0.660 | 6.62 | 35 |
| | HHHGG | | | |
| KJE05 | GGAAGGDPYGGDTRGGHPDGGFHSGG | 0.676 | 6.62 | 36 |
| | HHHHGG | | | |
| KJE06 | GGAAGGYFSGGETHGGHSPGGDFPGGH | 0.660 | 6.62 | 37 |
| | HHHGG | | | |
| KJE07 | GGAAGGHSPGGDFPGGDPYGGDTRGG | 0.675 | 6.48 | 38 |
| | HHHHGG | | | |
| KJE08 | GGAAGGHPDGGFHSGGYFSGGETHGGH | 0.742 | 6.73 | 39 |
| | HHHGG | | | |
| KJE09 | KGKAGGHSPGGDFPGGYFSGGFHSGGH | 0.632 | 9.99 | 40 |
| | HHHGG | | | |

What is claimed is:

1. A composition, comprising a set of different polypeptides,
wherein the set of different polypeptides comprises at least 3 different polypeptides comprising amino acid sequences of at least 10 amino acids,
wherein a set of different epitopes occurs in the set of different polypeptides, the set of different epitopes comprising at least 10 different epitopes, each of the different epitopes comprising at least 3 amino acids in the amino acid sequences of a subset of the different polypeptides, the subset of the different polypeptides comprising at least 2 different polypeptides, and
wherein the amino acid sequences of the different polypeptides each comprises a subset of different epitopes of the set of epitopes, the subset of the different epitopes comprising at least 3 epitopes.

2. The composition of claim 1, wherein the amino acid sequences each comprise at most 100 amino acids.

3. The composition of claim 1, wherein the set of different polypeptides comprises at least 10 different polypeptides comprising amino acid sequences of at least 20 amino acids .

4. The composition of claim 3, wherein the set of different epitopes comprises at least 50 different epitopes

5. The composition of claim 4, wherein the subset of the different epitopes comprises at least 10 epitopes.

6. The composition of claim 1, wherein the amino acid sequence for at least one of the different polypeptides is non-naturally occurring.

7. The composition of claim 1, wherein the amino acid sequences for at least two of the different polypeptides do not co-occur in any naturally occurring organism.

8. The composition of claim 1, wherein each of the different epitopes comprises at most 6 amino acids

9. The composition of claim 1, wherein the polypeptides are immobilized on a solid support.

10. The composition of claim 9, wherein the solid support comprises an array of addresses, wherein individual polypeptides of the set of standard polypeptides are each attached to a respective address of the array.

11. The composition of claim 1, wherein the set of different polypeptides comprises a polypeptide comprising the amino acid sequence of any one of SEQ ID NO: 1 to SEQ ID NO: 40.

12. The composition of claim 1, wherein a polypeptide of the set of different polypeptides is non-covalently bound to an antibody or functional fragment thereof.

13. A method of preparing a polypeptide sample, comprising
(a) obtaining a polypeptide extract from an organism;
(b) contacting the polypeptide extract with a set of standard polypeptides comprising the composition claim 1, thereby forming a polypeptide sample comprising polypeptides from the extract and the at least one standard polypeptide.

14. A method of detecting polypeptides, comprising
(a) obtaining a sample comprising a set of standard polypeptides comprising the composition of claim 1 and a plurality of test polypeptides from an organism; and
(b) detecting at least one polypeptide from the organism in the sample and detecting the at least one standard polypeptide in the sample.

15. A kit comprising the composition of claim 1.

16. The kit of claim 15, further comprising a plurality of affinity reagents that recognize epitopes of the set of different epitopes.

17. A flow cell comprising the composition of claim 1.

18. A detection apparatus comprising the flow cell of claim 17.

19. The detection apparatus of claim 18, further comprising one or more reservoirs containing a plurality of affinity reagents that recognize epitopes of the set of different epitopes.

20. A method of preparing a polypeptide sample, comprising
(a) obtaining a plurality of test polypeptides from an organism;
(b) contacting the plurality of test polypeptides with a standard polypeptide having the amino acid sequence of any one of SEQ ID NO: 1 to SEQ ID NO: 40, thereby forming a polypeptide sample comprising the plurality of test polypeptides and the at least one standard polypeptide.

21. A method of detecting polypeptides, comprising
(a) obtaining a polypeptide sample comprising test polypeptides from an organism and a standard polypeptide having the amino acid sequence of any one of SEQ ID NO: 1 to SEQ ID NO: 40; and
(b) detecting at least one of the test polypeptides in the sample and detecting the standard polypeptide in the sample.
